(19) United States
(12) Patent Application Publication
(10) Pub. No.: US 2007/0167377 A1
(43) Pub. Date: Jul. 19, 2007

(54) Inhibitors of glycinamide ribonucleotide transformylase

(76) Inventors: Dale L Boger, La Jolla, CA (US); Ian A Wilson, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 10/510,405

(22) PCT Filed: Apr. 7, 2003
(86) PCT No.: PCT/US03/10944

§ 371 Date: May 4, 2005

(30) Foreign Application Priority Data
Apr. 5, 2002 (US) 60370591

(51) Int. Cl. A61K 38/06; A61K 38/05; A61K 38/04; A61K 31/513

(52) U.S. Cl. 514/17; 514/18; 514/269; 530/330; 530/331; 544/314

(57) ABSTRACT

Potent human inhibitors of human glycinamide ribonucleotide transformylase and of aminoimidazole carboxamide ribonucleotide transformylase are designed, synthesized, and characterized.

### FIELD OF INVENTION

The present application relates to inhibitors of transformylases. More particularly, the present invention relates to inhibitors of glycinamide ribonucleotide transformylase and of aminoimidazole carboxamide ribonucleotide transformylase and their use.

### BACKGROUND

Glycinamide ribonucleotide transformylase (GAR Tfase) is a folate-dependent enzyme within the de novo purine biosynthetic pathway. GAR Tfase utilizes the cofactor 10-formyl-tetrahydrofolic acid (10-formyl-THF) in the third step of the pathway to transfer a formyl group to the primary amine of its substrate, β-glycinamide ribonucleotide (β-GAR). GAR Tfase is of mechanistic interest for the ease with which it catalyzes the formyl transfer, of biological interest for its role in the synthesis of DNA precursor purines, of structural interest for delineation of key mechanistic features of its catalytic reaction, and of medicinal interest as an important target for chemotherapeutic drug design.

Inhibitors of folate metabolism have provided important agents for cancer chemotherapy as a result of their inhibition of the biosynthesis of nucleic acid precursors (reviewed in Newell, D. R., Semin. Oncol. 1999, 26, 74-81; and Takimoto, C. H., Semin. Oncol. 1997, 24, A18-40-S18-51). Validation of GAR Tfase as an anti-cancer target came in the 1980's with the discovery of the first potent and selective inhibitor, 5,10-dideaza-5,6,7,8-tetrahydrofolic acid (DDATHF) (Taylor, E. C., et al., J. Med. Chem. 1985, 28, 914-921). This compound exhibits effective activity in vivo against solid murine and human tumors, where Methotrexate (MTX) has little effect. The selectivity of DDATHF has been attributed to reliance of tumor cells on de novo purine synthesis, while the salvage pathway is the primary source of purines in most normal cells. The (6R)-diastereomer, Lometrexol (LTX, Kᵢ=60 nM) (FIG. 13) has been in and out of clinical trials, initially as a consequence of its effective anti-neoplastic activity (Beardsley, G. P., et al., J. Biol. Chem. 1989, 264, 328-333) and, more recently, due to reduction of its general toxicity when supplemented with folic acid (Laohavinij, S., et al., Invest. New Drugs 1996, 14, 325-335; and Roberts, J. D., et al., Cancer Chemother. Pharmacol. 2000, 45, 103-110).

Human GAR Tfase (purN) is located at the C-terminus of a trifunctional enzyme encoded by purD-purM-purN with a molecular weight of more than 110 kD. The other two enzyme activities are GAR synthetase (purD) and AIR synthetase (purM), that represent steps 2 and 5 in the de novo purine biosynthetic pathway. Due to the complexity of the trifunctional enzyme, the majority of the biological and structural studies of GAR Tfase have been performed with the protein isolated from bacterial sources; the E. coli enzyme shares 31% overall sequence identity with its human counterpart, but that increases to almost 100% within the active site. The monofunctional E. coli GAR Tfase with a molecular weight of 23 kD has been a useful surrogate target for the human enzyme for mechanistic studies for many years, and more recently for inhibitor design (Varney, M. D., et al., J. Med. Chem. 1997, 40, 2502-2524; Boger, D. L., et al., Bioorg. Med. Chem. 1998, 6, 643-659; Boger, D. L., et al., Bioorg. Med. Chem. Lett. 2000, 10, 1471-1475; and Boger, D. L., et al., Bioorg. Med. Chem. 2000, 8, 1075-1086). However, an understanding of any subtleties in the activity and function of human versus bacterial GAR Tfase function has been hampered by the lack of any structural data for mammalian GAR Tfases. For example, the mammalian polyglutamation of the folate cofactor entails only γ-carboxylate linkages in the glutamate tail (Moran, R. G., Adv. Exp. Med. Biol. 1983, 163, 327-339; Shane, B., et al., J. Bacteriol. 1983, 153, 316-325; and McGuire, J. J., et al., Biochem. Pharmacol. 1984, 33, 1355-1361), whereas α and γ polyglutamation are observed in E. coli and other bacterial systems (Ferone, R., et al., J. Biol. Chem. 1986, 261, 16363-16371; and Ferone, R., et al., J. Biol. Chem. 1986, 261, 16356-16362); presumably the human and E. coli GAR Tfase structures should reflect such differences in their interaction with the polyglutamated tail.

Our recent structure of recombinant human GAR Tfase (rhGAR Tfase) revealed a number of important differences between the human and E. coli enzymes. Recombinant human GAR Tfase exists as a monomer at a wide range of pH values, in contrast to the dimerization observed for E. coli GAR Tfase below pH 6.8. The active site loop-helix (residues 110-131) that undergoes pH-dependent order-disorder transition in E. coli GAR Tfase has a uniform conformation under all pH ranges tested (pH 4-9) in the human enzyme. Although the substrate-binding pocket in E. coli GAR Tfase always adopts the same conformation under a wide range of pH conditions (pH 3.5-8), a loop (residues 8-14) in the human enzyme changes from an open to occluded conformation at low pH that appears to prohibit the substrate binding. Most importantly, the folate-binding loop, which intimately interacts with bound folate analogues, adopts different conformations in the unliganded human GAR Tfase from those described previously for the E coli enzyme.

Glycinamide ribonucleotide transformylase (GAR Tfase) is an enzyme central to de novo purine biosynthesis. Since purines are crucial components of DNA and RNA, inhibition of enzymes in the purine biosynthetic pathway has been proposed to be an effective approach for antineoplastic intervention (Divekar, A. Y., et al., Mol. Pharmacol. 1975, 11, 319; Moras, R. G. In Cancer Treatment and Research 1991, 58, 65; and Berman, E. M., et al., J. Med. Chem. 1991, 34, p 479). The disclosure that (6R)-5,10-dideazatetrahydrofolate (Lometrexol, (6R)-DDATHF) is an efficacious antitumor agent that acts as an effective inhibitor of GAR Tfase (Kᵢ=0.1 mM) established inhibition of purine biosynthesis and GAR Tfase as viable targets for antineoplastic intervention. GAR Tfase uses (6R)-10-formyl-5,6,7,8-tetrahydrofolate (1) to transfer a formyl group to the primary amine of its substrate, glycinamide ribonucleotide (2a, GAR; FIG. 1). This one carbon transfer constitutes the incorporation of the C-8 carbon of the purines and is the first of two formyl transfer reactions. The second formyl transfer reaction is catalyzed by aminoimidazole carboxamide ribonucleotide transformylase (AICAR Tfase) which also employs 1 to transfer a formyl group to the C-5 amine of its substrate, aminoimidazole carboxamide ribonucleotide (2b, AICAR; FIG. 1). (Warren, L., et al., J. Biol. Chem. 1957, 229, 613; Buchanan, J. M., et al., Adv. Enzymol. 1959, 21, 199; Flaks, J. G., et al., J. Biol. Chem. 1957, 229, 603; Flaks, J. G., et al., J. Biol. Chem. 1957, 228, 215; Warren, L., et al., J. Biol. Chem. 1957, 229, 627; Smith, G. K., et al., In Chemistry and Biology of Pteridins; Blair, J. A., Ed.; Walter de Gruyter: Berlin, 1983; pp 247-250; Baggott, J. E., et al., Biochemistry 1979, 18, 1036; Rayl, E. A., et al., J. Biol. Chem. 1996, 271, 2225; Ni, L., et al., Gene 1991, 106, 197; Chopra, A. K., et al., Biochim. Biophys. Acta 1991, 1090, 351; Szabados, E., et al., Biochemistry 1994, 33, 14237; Mueller, W. T., et al., Biochemistry 1981, 20, 337; Aiba, A., et al., J. Biol. Chem. 1989, 264, 21239; and Ebbole, D. J., et al., J. Biol. Chem. 1987, 262, 8274). Herein, we detail the preparation and evaluation of 10-formyl-DDACTHF (3) in our continued efforts to identify potent inhibitors of GAR Tfase and AICAR Tfase.

In previous studies, aldehyde containing folate-based inhibitors incapable of transferring the formyl group were analyzed. (Boger, D. L., et al., Bioorg. Med. Chem. 1997, 5, 1817; Boger, D. L., et al., Bioorg. Med. Chem. 1997, 5, 1831; Boger, D. L., et al., Bioorg. Med. Chem. 1997, 5, 1839; Boger, D. L., et al. Bioorg. Med. Chem. 1997, 5, 1847; Boger, D. L., et al., Bioorg. Med. Chem. 1997, 5, 1853; Boger, D. L., et al., Bioorg. Med. Chem. 1998, 6, 643; Boger, D. L., et al., Bioorg. Med. Chem. 2000, 8, 1075; and Boger, D. L., et al., Bioorg. Med. Chem. Lett. 2000, 10, 1471). Thus, replacement of N10 with a carbon atom prevents the transfer of the formyl group from the cofactor analogue providing unique opportunities for enzyme inhibition. This could entail either competitive inhibition of the enzymes through gem-diol binding of the aldehyde mimicking the formyl transfer tetrahedral intermediate or covalent trap of the substrate at the active site to provide enzyme-assembled tight binding inhibitors of GAR or AICAR Tfase. (Li, S. W., et al., Med. Chem. Res. 1991, 1, 353; For related studies with 5-DACTHF, see: Bigham, E. C., et al., Heterocycles 1993, 35, 1289; Inglese, J., et al., J. Med. Chem. 1989, 32, 937; and Inglese, J., et al., Tetrahedron 1991, 47, 2351). Co-crystallization of GAR Tfase, β-GAR and 10-formyl-5,8,10-trideazafolate (10-formyl-TDAF), the most potent of the inhibitors examined to date, revealed that the aldehyde inhibitor (Kᵢ=260 nM) binds in the active site as its hydrate mimicking the tetrahedral intermediate involved in formyl transfer (Greasley, S. E., et al., Biochemistry 1999, 38, 16783). Thus, no enzyme-assembled imine adduct with the substrate β-GAR or covalent adduct with nucleophiles of the GAR Tfase active site residues were observed, and the potent inhibitory activity could be attributed to the H-bonding interactions of the inhibitor aldehyde hydrate with the catalytically important residues of the enzyme active site. Despite these efforts, none of the potent GAR Tfase inhibitors in this series, including 10-formyl-TDAF, exhibited cytotoxic activity consistent with their level of enzyme inhibition potency; observations that could be attributed in part to their instability and ineffective transport by the reduced folate carrier.

Numerous reports have described acyclic analogues of (6R)-5,10-dideazatetrahydrofolate (4, Lometrexol or (6R)-DDATHF, FIG. 2). (Baldwin, S. W., et al., Biochemistry 1991, 30, 1997; Sokoloski, J. A., et al., Cancer Chemother. Pharmacol. 1991, 28, 39; Shih, C., et al., J. Med. Chem. 1992, 35, 1109; Bigham, E. C., et al., J. Med. Chem. 1992, 35, 1399; Taylor, E. C., et al., J. Org. Chem. 1992, 57, 3218; Mullin R. J., et al., Biochem. Pharmacol. 1992, 43, 1627; Jansen, M., et al., Biochem. Pharmacol. 1994, 47, 1067; and Habeck, L. L., et al., Mol. Pharmacol. 1995, 48, 326). Several of these analogues, including the acyclic derivative 5 (FIG. 2, X═CH₂) of DDATHF, (Taylor, E. C., et al., Heterocycles 1989, 28, 1169) have been shown to retain the potent cytotoxic and enzyme inhibitory properties of 4. Additionally, several analogues of 4 with substituents at C-10 (e.g. 10-methyl and 10-hydroxymethyl) exhibit equivalent or increased biological activity relative to 4. (Taylor, E. C., et al., Tetrahedron 1992, 48, 19).

### SUMMARY

Initial Inhibitors:

A series of initial compounds were synthesized and evaluated as potential inhibitors of GAR Tfase and AICAR Tfase. Four compounds (3, 14, 15, and 17) were identified as having potent biological activity (IC₅₀ values less than 0.20 mM) in the absence of media purines, indicating selective cytotoxicity through the inhibition of the purine de novo biosynthetic pathway. Purine and AICAR rescue experiments indicate that they exhibit their potent cytotoxic activity specifically through intracellular GAR Tfase inihibition even though none of the compounds examined demonstrated sub-micromolar in vitro inhibition of E. coli GAR Tfase or human AICAR Tfase.

Subsequent assays were performed in order to determine if polyglutamation and/or reduced folate carrier transport were responsible for the significant increase in cellular biological activity compared to in vitro enzymatic activity. The lack of cytotoxic activity of agents (3, 14, 15, and 17) against CCRF-CEM cells with impaired reduced folate active transport (CCRF-CEM/MTX) indicates that these agents require the reduced folate carrier for biological activity and their inactivity against CCRF-CEM/FPGS⁻ lacking folylpolyglutamate synthase establishes that their polyglutamation is also required for activity. The γ-pentaglutamate derivatives 21 and 22 demonstrated only marginal enhanced binding affinity for E. coli GAR Tfase, and a more significant 4′ (21) and 140′ (22) enhanced binding affinity for human AICAR Tfase resulting in inhibitors with a 10′ higher affinity for human AICAR Tfase over E. coli GAR Tfase in vitro. These observations on the pentaglutamates, while interesting, were inconsistent with GAR Tfase as a primary site of action. Subsequent examination of the inhibitors against human GAR Tfase revealed that they and the corresponding γ-pentaglutamates were unexpectedly much more potent against the human versus E. coli enzyme which also contributes to their exceptional cytotoxic potency.

Advanced Inhibitors:

Here, we disclose the use of a structure-based approach to design an advanced folate analogue, viz., 10-trifluoroacetyl-5,10-dideaza-acyclic-5,6,7,8-tetrahydrofolic acid (10-CF₃CO-DDACTHF, 101), which specifically inhibits recombinant human GAR Tfase (Kᵢ=15 nM), but is inactive (Kᵢ>100 μM) against other folate-dependent enzymes examined. Moreover, compound 101 is a potent inhibitor of tumor cell proliferation (IC₅₀=16 nM, CCRF-CEM), which represents a 10-fold improvement over Lometrexol, a GAR Tfase inhibitor that has been in clinical trials. Thus, this folate analogue 101 is among the most potent and selective inhibitors known towards GAR Tfase. Contributing to its efficacious activity, compound 101 is effectively transported into the cell by the reduced folate carrier and intracellularly sequestered by polyglutamation. The crystal structure of human GAR Tfase with folate analogue 101 at 1.98 Å resolution represents the first structure of any GAR Tfase to be determined with a cofactor or cofactor analogue without the presence of substrate. The folate-binding loop 141-146, which shows high flexibility in both E. coli and unliganded human GAR Tfase structures, becomes highly ordered upon binding 101 in the folate-binding site. Computational docking of the natural cofactor into this and other folate analogue-substrate bound structures provides a rational basis to model how the natural cofactor 10-formyl-tetrahydrofolic acid interacts with GAR Tfase, and suggests that this folate analogue bound conformation represents the best template to date for inhibitor design.

One aspect of the invention is directed to a compound represented by the following structure: 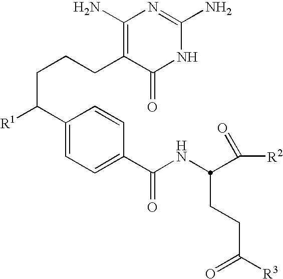
In the above structure, R¹ is a radical selected from the group consisting of —C(O)H, —CH₂OH, —CH═NNMe₂, —C(O)CF₃, and —CH(OH)CF₃; R² is a radical selected from the group consisting of —OH, —OtBu, glutamyl, and oligoglutamyl; R³ is a radical selected from the group consisting of —OH, —OtBu, glutamyl, and oligoglutamyl; each glutamyl being independently represented by the formula —NHCH(C(O)R⁴)(CH₂)₂C(O)R⁵, wherein R⁴ and R⁵ are each radicals independently selected from the group consisting of —OH and —OtBu; each oligoglutamyl having at least one terminal glutamyl and between one and four non-terminal glutamyl residues; each terminal glutamyl being independently represented by the formula —NHCH(C(O)R⁴)(CH₂)₂C(O)R⁵, wherein R⁴ and R⁵ are each radicals independently selected from the group consisting of —OH and —OtBu; each non-terminal glutamyl being independently represented by the formula —NHCH(C(O)R⁶)(CH₂)₂C(O)R⁷, wherein R⁶ and R⁷ are each radicals independently selected from the group consisting of —OH, —OtBu, terminal glutamyl, and non-terminal glutamyl; with a proviso that at least one of R⁶ and R⁷ is either terminal glutamyl or non-terminal glutamyl. In two of the preferred embodiments of the invention, the compound is represented by the following structures:

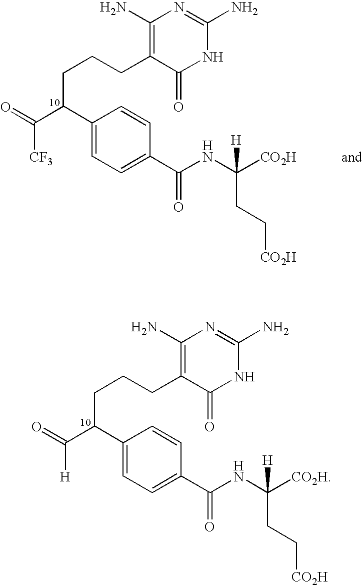

In a further preferred embodiment, the compound according to claim 1 represented by the following structure: 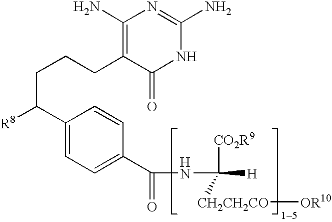
In the above structure, R⁸ is a radical selected from the group consisting of —C(O)H and —C(O)CF₃; and R⁹ and R¹⁰ are each a radical independently selected from the group consisting of —H and -tBu.

In a further preferred embodiment, the compound according to claim 1 represented by the following structure: 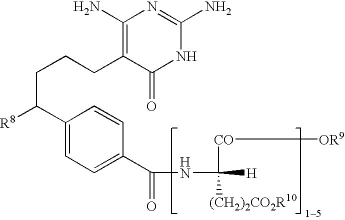
In the above structure, R⁸ is a radical selected from the group consisting of —C(O)H and —C(O)CF₃; and R⁹ and R¹⁰ are each a radical independently selected from the group consisting of —H and -tBu.

Another aspect of the invention is directed to a process for inhibiting glycinamide ribonucleotide transformylase comprising the step of contacting the glycinamide ribonucleotide transformylase with an inhibiting concentration of any of the compounds described above.

Another aspect of the invention is directed to a process for aminoimidazole carboxamide ribonucleotide transformylase comprising the step of contacting the aminoimidazole carboxamide ribonucleotide transformylase with an inhibiting concentration of any of the compounds described above.

The work presented herein represents a complete structure-based drug design cycle for GAR Tfase: structure, analysis, synthesis, and evaluation that then returns to structure. The structure of E. coli GAR Tfase in complex with the cofactor analogue 10-formyl-TDAF and substrate β-GAR (PDB code 1C2T) reveals that the inhibitor binds as a hydrated gem-diol, interacting with the enzyme in a manner that mimics the formyl transfer intermediate (Greasley, S. E., et al., Biochemistry 1999, 38, 16783-16793). Based on this structural insight, a new compound 10-CF₃CO-DDACTHF (101) was designed and synthesized to facilitate and stabilize the formation of a gem-diol in the binding site. The newly designed compound was found to be a selective and unusually effective inhibitor of rhGAR Tfase, representing the most potent folate analogue described to date. In addition, 101 was inactive against AICAR Tfase, TS and DHFR. This compound acts as a surrogate cofactor, but is incapable of formyl transfer. Its structural resemblance to the natural folate cofactor suggested that it might be accepted as a substrate for cellular folate transport systems, as well as for FPGS, as confirmed by cytotoxic assays. Most importantly, the compound is chemically stable. All these properties make this compound a potential lead for in vivo studies as a chemotherapeutic agent.

This compound was crystallized with rhGAR Tfase and its structure compared to an E coli structure with a related folate analogue, 10-formyl-TDAF (103) (Greasley, S. E., et al., Biochemistry 1999, 38, 16783-16793). The folate-binding loop of these two structures are very similar with a few minimal differences for the side chains of Glu141, Asp142 and Val143, most likely caused by the higher flexibility of this loop in the E. coli structure. Although other important differences have been found between the structure of bacterial and human GAR Tfases, the folate-binding pocket conformations share a high degree of resemblance so that information obtained from this particular E. coli GAR complex was invaluable for inhibitor design.

The availability now of unliganded and inhibitor-bound human GAR Tfase structures at high resolution reveal subtle changes upon inhibitor binding. The most dramatic change takes places in the folate-binding loop 141-146, which forms key interactions with the inhibitor. This conformational change juxtaposes Asp144 next to the tightly anchored His108 and Asn106. As His108 and Asn106 have consistent locations and conformations in various structures, the translocation of Asp144 upon inhibitor binding finally assembles the complete reaction triad within one structure into a configuration that is presumably ready for catalysis and proton shuttling in the formyl transfer reaction.

The coordinates of rhGAR Tfase/10-CF₃CO-DDACTHF (101) provide a much better model than apo human GAR Tfase for docking simulations of the natural folate cofactor. The less favorable docking energy to the apo protein and the inappropriate positioning of the folate cofactor in this structure suggest that the folate-binding loop undergoes a conformational change to accommodate the folate/folate analogue, probably by induced fit. For both human and E. coli GAR Tfases, one particular conformation of the folate binding loop is found to be the most energetically favorable, suggesting that the crystal structure of rhGAR Tfase in complex with 10-CF₃CO-DDACTHF provides a reasonable “snapshot” for its interaction with the natural folate cofactor. These combined crystallographic and computational studies greatly enhance our understanding of the GAR Tfase formyl transfer mechanism and the design of subsequent generations of GAR Tfase inhibitors.

### BRIEF DESCRIPTION OF DRAWINGS

FIG. 1 illustrates a scheme showing the reaction catalyzed by GAR Tfase in the biosynthesis of purines. There are two formyl transfer reactions in the biosynthetic pathway. The second formyl transfer is accomplished by aminoimidazole carboxamide ribonucleotide transformylase (AICAR Tfase).

FIG. 2 illustrates the structures of Lometrexol (4), (6R)-5,10-dideazatetrahydrofolate ((6R)-DDATHF), and the acyclic derivative 5 which is an analog of 4.

FIG. 3 illustrates a scheme showing the synthesis of 10-formyl-DDACTHF 3 which is an analog of 1 that bears a non-transferable formyl group.

FIG. 4 illustrates a scheme showing the formation of both 12 and 13 from 11. 13 is a side product that occurs as a result of oxidative deformylation in this reaction to hydrolyze the dimethyl-hydrazone.

FIG. 5 illustrates a scheme showing the production of the known alcohol 17 from aldehyde 12 using a standard set of reactions.

FIG. 6 illustrates a scheme showing the synthesis of folate analogs having γ-pentaglutamate linkage. This would establish the importance of the nature of this linkage for these compounds. Only γ-polyglutamates have been found in eukaryotes.

FIG. 7 illustrates a scheme showing the synthesis of folate analogs having the α-pentaglutamate linkage.

FIG. 8 illustrates a table showing GAR Tfase, AICAR Tfase, and DHFR inhibition with the selected compounds. The concentration is in μM.

FIG. 9 illustrates a table showing the results of testing compounds 3, 9-12, 14, 15, 17, 21, 22, 25, and 26 for in vitro cytotoxic activity both in the presence (+) and the absence (−) of added hypoxanthine against the CCRF-CEM cell line.

FIG. 10 illustrates a table showing the results of testing the featured compounds for in vitro cytotoxic activity in the presence of AICAR.

FIG. 11 illustrates two tables showing the lack of potency these compounds had with respect to the two different cell lines. The mutant CCRF-CEM cell line (CEM/MTX) has been shown to have an impaired reduced folate carrier. The lack of activity against this cell line indicates that having reduced folate carrier transport is essential for the analogs' biological activity. The second table shows the mutant CCRF-CEM cell line (CEM/FPGS₋) that lacks folylpolyglutamate synthase (FPGS). All the potent inhibitors including 3 and 15 lost cytotoxic activity against this cell line which indicates that the inhibitors are dependent on polyglutamation for their biological activity.

FIG. 12 illustrates a table showing the activity of the compounds against two different enzymes, recombinant human GAR Tfase and E. coli Tfase.

FIG. 13 illustrates the structure of an advanced GAR Tfase inhibitor, 101, along with the natural cofactor 10-formyl-THF and other inhibitors.

FIG. 14 illustrates a table showing the data collection and refinement statistics which were used on the data obtained from the single crystal of human GAR Tfase in complex with 10-CF₃CO-DDACTHF (101) examined at the Stanford Synchrotron Radiation Laboratory. ¹Numbers in parentheses refer to the highest resolution shell. ²R_{sym}=[Sₕ, Sᵢ|Iᵢ(h)−<I(h)>|/SₕSᵢIᵢ(h)]×100, where <I(h)> is the mean of the I(h) observation of reflection. ³R_{cryst}=Sₕ||Fₒ|−|F_{c}||/Sₕ|Fₒ| where Fₒ and F_{c} are the observed and calculated structure factor amplitudes. ⁴R_{free} (%) is the same as ³R_{cryst}, but for 5% of the data randomly omitted from the refinement.

FIG. 15 illustrates a scheme showing the steps used in the synthesis of compound 101. This scheme is analogous to the scheme shown in FIG. 3.

FIG. 16 illustrates a table showing the inhibition of E coli GAR Tfase, rhGAR Tfase and rhAICAR Tfase by the five compounds.

FIG. 17 illustrates a table showing the IC₅₀'s of the selected compounds against the mutant cell line CCRF-CEM which has impaired reduced folate transport across the cellular membrane.

FIG. 18A. illustrates a stereoview of rhGAR Tfase cocrystallized with 101 at physiological pH 7. FIG. 18A is a closer stereoview of the inhibitor bound in the folate binding site. Inhibitor, catalytic residues and ordered water molecules in the inhibitor binding site are illustrated in ball-and-stick using the same color scheme as in 18A.

FIG. 19 illustrates a table showing the B value comparison of unliganded human GAR Tfase, E. coli GAR Tfase in complex with 10-formyl-TDAF and substrate, and human GAR Tfase in complex with 10-CF₃CO-DDACTHF (101).

FIG. 20 illustrates four separate pictures of the Human GAR Tfase-10-CF₃CO-DDACTHF (101) interaction. 20A shows the final refined model of inhibitor 101 superimposed on the 2Fₒ-F_{c} electron density contoured at 2s. The key interactions of the inhibitor and human GAR Tfase are between the protein side chains and three moieties of the inhibitor: diaminopyrimidinone ring, trifluoroacetyl group and benzoyl-glutamate tail. 20B shows the interaction between the diaminopyrimidinone ring of the inhibitor 101 and GAR Tfase. The potential hydrogen bonds are drawn using dashed lines with the distances in Å.

FIG. 21 illustrates a picture showing the orientation of the glutamate tails of the folate analogs in complex with E. coli and human GAR Tfase. The translucent solvent accessible surface is superimposed on the ribbon diagram of the protein. FIG. 21A shows the preferred conformation for α-polyglutamated forms. The structure shown represents the complex structure between E. coli GAR Tfase with 10-formyl-TDAF and β-GAR (PDB code 1C2T). A salt bridge is formed between the Arg64 and the γ-carboxylate, so that the α-carboxylate is exposed to solvent. FIG. 21B shows the preferred conformation for γ-polyglutamated forms. The structure shown represents the human GAR Tfase complex with 101. The salt bridge is now between the Arg64 and the α-carboxylate, so that its γ-carboxylate is exposed to solvent.

FIG. 22 illustrates a series of four stereoviews of the GAR Tfase folate-binding loop 141-146. FIG. 22A shows the structural isomerism of the folate-binding loop 141-146. The 141-146 loops from different E. coli and human structures are superimposed onto the human GAR Tfase/101 complex with Asp144 shown in ball and stick. The inhibitor 10-CF₃CO-DDACTHF (101) is represented by ball and stick. FIG. 22B shows that the folate-binding loop in human GAR Tfase becomes ordered upon inhibitor 101 binding. The 2Fₒ-F_{c} electron density map of the loop is contoured at 2s with refined coordinates superimposed in ball-and-stick. FIG. 22C shows the docking interaction of the natural cofactor folate with human GAR Tfase. The catalytic triad (Asn106, His108 and Asp144) are in close proximity to the formyl group of the cofactor to facilitate the formyl transfer reaction. FIG. 22D shows the superposition of human GAR Tfase (human GAR Tfase/101 as template) and E. coli GAR Tfase (PDB code 1C2T) docked with the natural folate cofactor. The lowest energy clusters in both cases share substantial similarities in both protein and cofactor conformation.

FIG. 23 illustrates a table of the computational docking of folate cofactor into human and E. coli GAR Tfase structures.

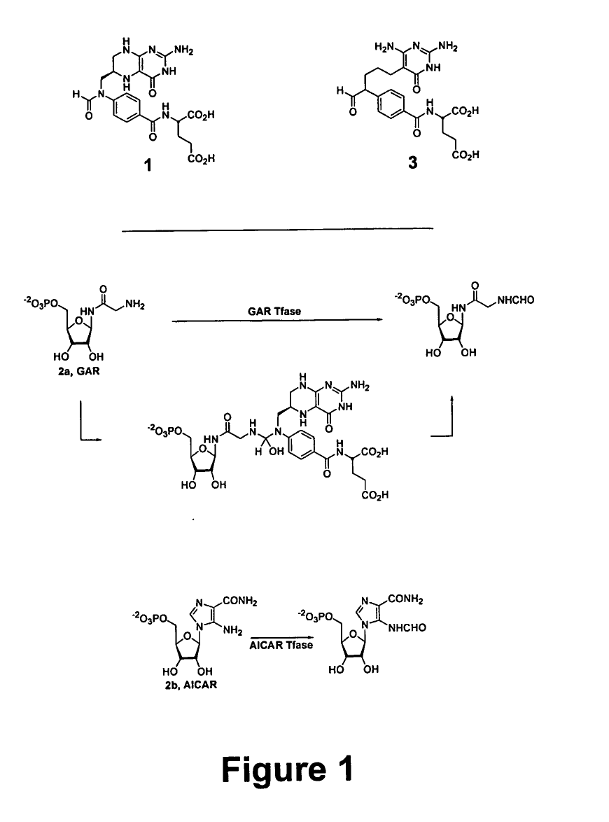

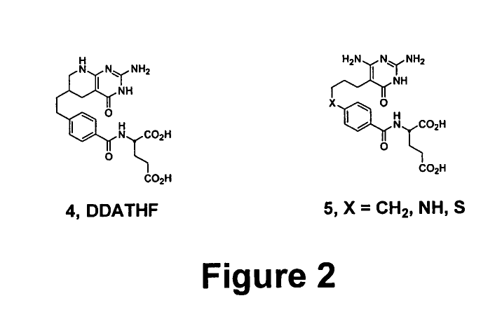

### DETAILED DESCRIPTION

### Initial Inhibitors

The synthesis of 10-formyl-DDACTHF (3) as a potential inhibitor of glycinamide ribonucleotide transformylase (GAR Tfase) and aminoimidazole carboxamide ribonucleotide transformylase (AICAR Tfase) is disclosed herein. Aldehyde 3, the corresponding γ- and α-pentaglutamates 21 and 25 and related agents were evaluated for inhibition of folate-dependent enzymes including GAR Tfase and AICAR Tfase. The inhibitors were found to exhibit potent cytotoxic activity (CCRF-CEM IC₅₀ for 3=60 nM) that exceeded their enzyme inhibition potency (Kᵢ (3)=6 and 1 mM for E. coli GAR and human AICAR Tfase, respectively). Cytotoxicity rescue by medium purines, but not pyrimidines, indicated that the potent cytotoxic activity is derived from selective purine biosynthesis inhibition and rescue by AICAR monophosphate established that the activity is derived preferentially from GAR versus AICAR Tfase inhibition. The potent cytotoxic compounds including aldehyde 3 lost activity against CCRF-CEM cell lines deficient in the reduced folate carrier (CCRF-CEM/MTX) or folylpolyglutamate synthase (CCRF-CEM/FPGS⁻) establishing that their potent activity requires both reduced folate carrier transport and polyglutamation. Unexpectedly, the pentaglutamates displayed surprisingly similar Kᵢ's versus E. coli GAR Tfase and only modestly enhanced Kᵢ's versus human AICAR Tfase. On the surface this initially suggested that the potent cytotoxic activity of 3 and related compounds might be due simply to preferential intracellular accumulation of the inhibitors derived from effective transport and polyglutamation (i.e., ca. 100-fold higher intracellular concentrations). However, a subsequent examination of the inhibitors against recombinant human GAR Tfase revealed they and the corresponding γ-pentaglutamates were unexpectedly much more potent against the human versus E. coli enzyme (Kᵢ for 3, 14 nM against rhGAR Tfase versus 6 mM against E. coli GAR Tfase) which also accounts for their exceptional cytotoxic potency.

Chemistry.

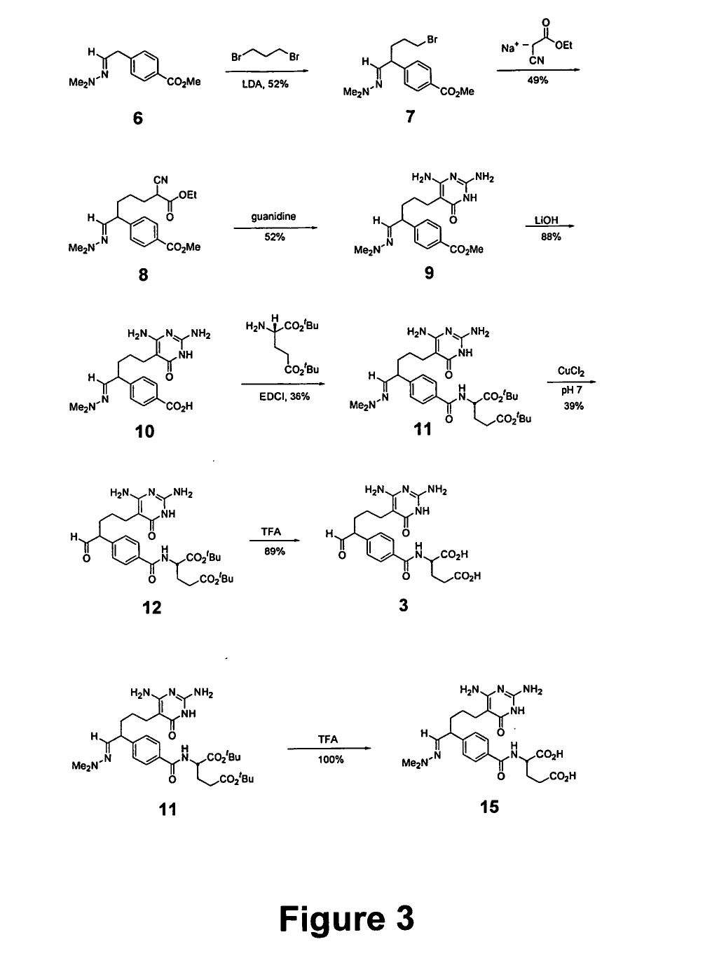

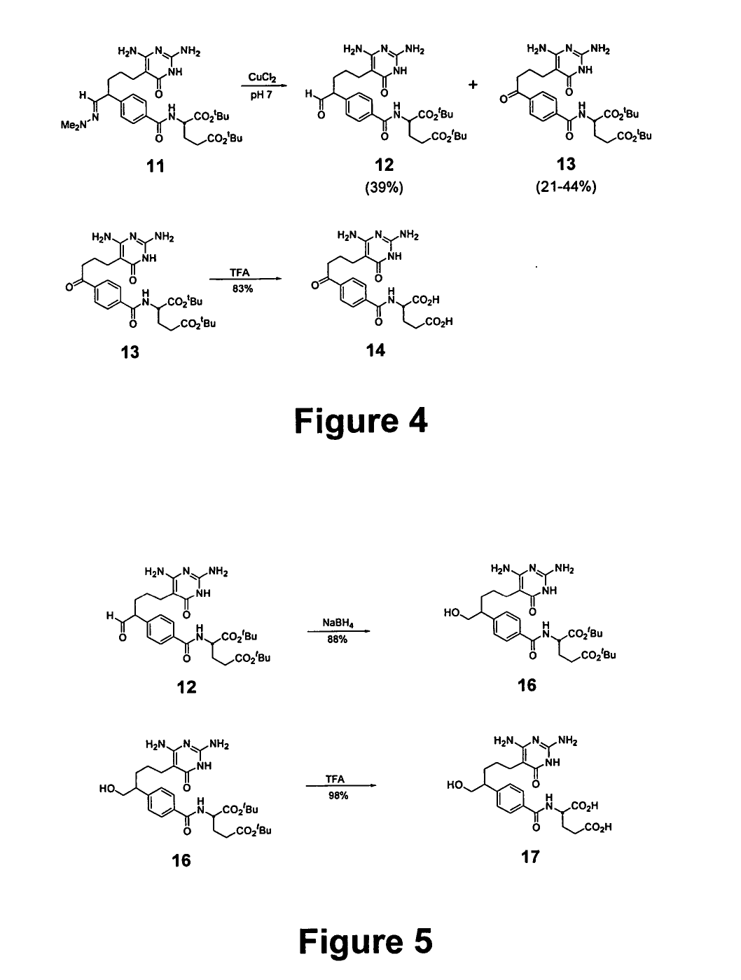

The synthesis of 10-formyl-DDACTHF (3) was accomplished in a convergent manner through alkylation (Corey, E. J., et al., Tetrahedron Lett. 1976, 3; Corey, E. J., et al., Chem. Ber. 1978, 111, 1337; and Corey, E. J., et al., Chem. Ber. 1978, 111, 1362) of the known N,N-dimethylhydrazone 6 with 1,3-dibromopropane. LDA deprotonation of 6 (THF, −78° C., 30 min) and subsequent treatment with excess 1,3-dibromopropane (10 equiv, HMPA, −78° C., 2 h, 52%) provided the key intermediate 7 (FIG. 3). The pre-formed sodium salt of ethyl cyanoacetate (NaH, DMF, 0° C., 30 min) was alkylated with 7 (DMF, 25° C., 2.5 h, 49%) providing 8. Cyclization with the free base of guanidine (1.1 equiv, CH₃OH, 25° C., 12 h, 52%) under basic conditions gave the desired pyrimidine 9. Treatment of 9 with LiOH (3.0 equiv, 3:1 CH₃OH—H₂O, 25° C., 12 h, 88%) cleanly provided the carboxylic acid 10 which was coupled with di-tert-butyl L-glutamate hydrochloride (EDCI, NaHCO₃, DMF, 25° C., 12 h) to provide 11. Subsequent hydrolysis of the dimethylhydrazone was accomplished to provide the sensitive aldehyde 12 by treatment with CuCl₂ (5.0 equiv, 0° C., 1 h, 39%) in THF—H₂O buffered to pH 7. In addition to obtaining aldehyde 12, the oxidative deformylation product 13 (21-44%, FIG. 4) was also obtained. Deprotection of 12 was accomplished by treatment with trifluoroacetic acid (1:5 v/v TFA/CHCl₃, 12 h, 89%) to provide 10-formyl-DDACTHF (3). Acid-catalyzed deprotection of 13 by treatment with trifluoroacetic acid (10 equiv, CHCl₃, 12 h, 83%) provided 14 (FIG. 4).

In addition, the stable N,N-dimethylhydrazone 11 was also converted to 15 by acid-catalyzed deprotection of the di-tert-butyl esters by treatment with trifluoroacetic acid (1:4 v/v TFA/CHCl₃, 12 h, quantitative) (FIG. 3). For comparative purposes, the aldehyde 12 was reduced to the alcohol 16 with NaBH₄ (3.0 equiv, CH₃OH, 4 h, 88%) followed by deprotection of 16 with trifluoroacetic acid (1:10 v/v TFA/CHCl₃, 12 h, 98%) to provide the known alcohol 17 (Taylor, E. C., et al., Tetrahedron 1992, 48, 19) (FIG. 5).

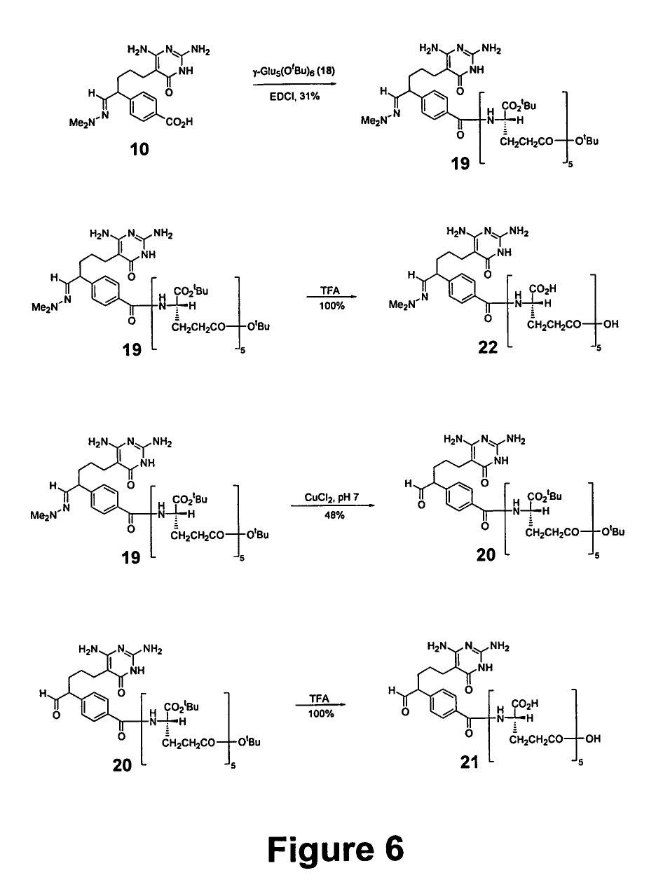

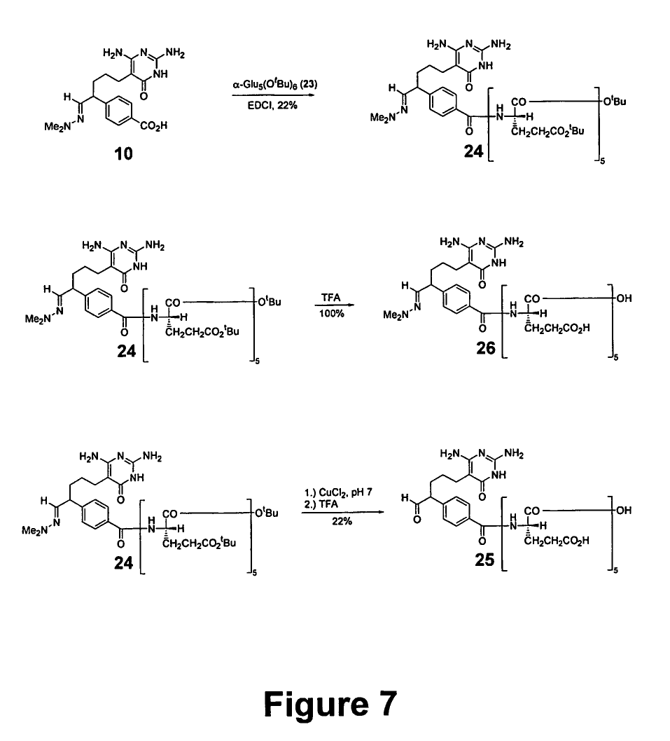

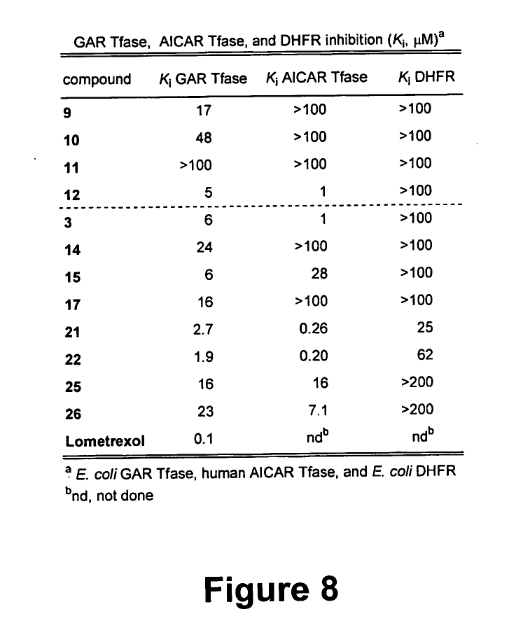

In efforts to establish the origin of the potent cytotoxic activity of 3 and related compounds, the corresponding γ- and α-pentaglutamates of 3 and 15 were also prepared. Whereas only γ-polyglutamates have been found in eurkaryotes, bacteria including E. coli produce folate conjugates which contain two γ and subsequent a glutamate linkages (i.e. pAB(γ-Glu)₂-(α-Glu)ₙ) (Ferone, R., et al., J. Biol. Chem. 1986, 261, 16356; and Ferone, R., et al., J. Biol. Chem. 1986, 261, 16363). To establish the importance of the nature of the linkage, both the γ- and α-pentaglutamates were prepared. The carboxylic acid 10 was coupled with the known free amine of the tert-butyl ester protected γ-pentaglutamate 18 (Styles, V. L., et al., J. Heterocyclic Chem. 1990, 27, 1809) (EDCI, NaHCO₃, DMF, 25° C., 48 h, 31%) to provide 19 (FIG. 6) as well as with the known free amine of the tert-butyl ester protected α-pentaglutamate 23 (Reig, F., et al., Invest. Inf. Text. Tensioactivos 1978, 21, 337; and Marin, O., et al., Int. J. Peptide Protein Res. 1990, 36, 374) (EDCI, NaHCO₃, DMF, 25° C., 48 h, 22%) to provide 24 (FIG. 7). Subsequent hydrolysis of the dimethylhydrazone in 19 was accomplished to generate the sensitive aldehyde 20 by treatment with CuCl₂ (5.0 equiv, 0° C., 1 h, 48%) in THF—H₂O buffered to pH 7. Subsequent deprotection of the tert-butyl esters was accomplished by treatment with trifluoroacetic acid (1:4 v/v TFA/CHCl₃, 12 h, 100%) to provide the 10-formyl-DDACTHF γ-pentaglutamate 21. In a similar manner, hydrolysis of the dimethylhydrazone in 24 was accomplished to generate the corresponding sensitive aldehyde by treatment with CuCl₂ (5.0 equiv, 0° C., 1 h) in THF—H₂O buffered to pH 7. Subsequent deprotection of the tert-butyl esters was accomplished by treatment with trifluoroacetic acid (1:4 v/v TFA/CHCl₃, 12 h, 22% from 24) to provide the 10-formyl-DDACTHF α-pentaglutamate 25. Similarly, the N,N-dimethylhydrazones 19 and 24 were converted to 22 and 26, respectively, by acid-catalyzed deprotection of the di-tert-butyl esters (1:4 v/v TFA/CHCl₃, 12 h, 100%) for direct comparison (FIGS. 6 and 7).

GAR Tfase, AICAR Tfase, and DHFR Inhibition.

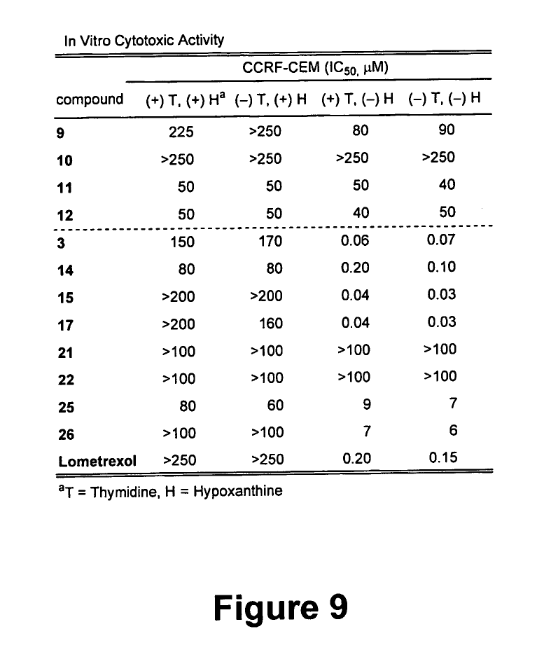

Compounds 3, 9-12, 14, 15, 17, 21, 22, 25, and 26 were tested initially for inhibition of E. coli GAR Tfase, human AICAR Tfase, and E. coli DHFR, and the results are presented in FIG. 9. With the exception of 11, all compounds demonstrate inhibition of GAR Tfase within one order of magnitude Kᵢ range (1.9-48 mM). Compounds 3 and 12 were also found to be effective inhibitors of AICAR Tfase with identical Kᵢ values of 1 mM, surprisingly comparable to the Kᵢ's observed with GAR Tfase. This lack of potentiation by the glutamate (3 vs 12) suggests that the enzyme inhibition properties are being dominated by the presence of the aldehyde in 3 and 12. This unusual observation is further discussed below in connection with human GAR Tfase. A more conventional glutamate potentiation of the dimethylhydrazone 15 (vs 11) was observed. Although the GAR Tfase inhibition by 15 proved comparable to that of the aldehyde 3 (Kᵢ=6 mM), 15 was 30-fold less effective than 3 against AICAR Tfase. Also representative of this importance of the aldehyde in 3, the corresponding alcohol 17 and the norketone 14 were significantly less active against GAR Tfase and inactive against AICAR Tfase. Similar observations have been made with related series of aldehyde-based inhibitors (CHO>HC═NNMe₂>CH₂OH>C═O) with the exception that the inhibition of GAR Tfase has generally been greater than AICAR Tfase. While this was observed with 14, 15, and 17, the aldehyde 3 was found to be slightly more potent against AICAR Tfase.

Interestingly, the aldehyde γ-pentaglutamate 21 and dimethylhydrazone γ-pentaglutamate 22 did not exhibit as large an increase in affinity for GAR Tfase as expected. Both γ-pentaglutamate derivatives only exhibit a 2-3′ higher binding affinity for E. coli GAR Tfase as compared to the monoglutamate inhibitors. A similar modest 4-fold increase in potency was observed with the aldehyde γ-pentaglutamate 21 versus 3 against AICAR Tfase, whereas the dimethylhydrazone 22 exhibited a much more substantial 140-fold increase relative to 15. Moreover, both γ-pentaglutamate derivatives exhibit an ca. 10× higher binding affinity for AICAR Tfase than GAR Tfase.

Since it has been shown previously that there are two folylpolyglutamate synthetase activities (both α and γ) in E. coli, (Ferone, R., et al., J. Biol. Chem. 1986, 261, 16356; and Ferone, R., et al., J. Biol. Chem. 1986, 261, 16363) the α-pentaglutamate derivatives 25 and 26 were also synthesized and evaluated. The aldehyde α-pentaglutamate 25 was 3-16′ less potent than the aldehyde monoglutamate 3 against GAR Tfase and AICAR Tfase, respectively. Likewise, the dimethylhydrazone α-pentaglutamate 26 was 4′ less potent than the hydrazone monoglutamate 15 against GAR Tfase, whereas it was 4′ more potent against AICAR Tfase and both were 1-2 orders of magnitude less potent than the corresponding γ-pentaglutamate 22. Thus, the γ-pentaglutamates were notably more potent than the α-pentaglutamates. However, with the exception of 22 versus AICAR Tfase, the γ-pentaglutamates were not significantly more potent enzyme inhibitors than the corresponding monoglutamates. While interesting, this behavior toward E. coli GAR Tfase proved not to be consistent with the functional potency of the compounds.

None of the compounds tested for inhibition of DHFR exhibited activity, establishing a selectivity for GAR Tfase and AICAR Tfase versus DHFR.

Cytotoxic Activity.

Compounds 3, 9-12, 14, 15, 17, 21, 22, 25, and 26 were examined for cytotoxic activity both in the presence (+) and absence (−) of added hypoxanthine against the CCRF-CEM cell line (FIG. 9). The cytotoxic activity of the precursor agents (9-12) was relatively nonpotent and uniform against the CCRF-CEM cell line regardless of whether the assay was conducted in the presence or absence of a media purine (hypoxanthine) or pyrimidine (thymidine). In contrast, aldehyde 3, like Lometrexol, exhibits no activity against the CCRF-CEM cell line cultured in media supplemented with a purine. However, both Lometrexol and aldehyde 3 exhibit potent cytotoxic activity (IC₅₀=0.15 mM and 0.06-0.07 mM, respectively) when purines are absent in the media. This sensitivity to the presence of purines, but not pyrimidines, indicates that the activity of aldehyde 3 is derived from its inhibition of enzymes in the de novo purine biosynthetic pathway.

Significantly, the degradation product 14, obtained by oxidative deformylation of 3, proved to be only slightly less potent or roughly equivalent in potency (IC₅₀=0.10 mM) to 3 and it also exhibited the selective purine rescue. Even more significantly, both the stable N,N-dimethylhydrazone 15 and the alcohol 17 exhibited a purine sensitive cytotoxic potency (IC₅₀=0.03 mM and 0.03 mM, respectively) that was at least as great or exceeded that of the aldehyde 3 or ketone 14. In each case with 3, 14, 15, or 17, the reversal of the cytotoxicity with hypoxanthine (100 mM) resulted in a ³10³-10⁴ change in the IC₅₀ value indicating that the activity was being observed through selective inhibition of purine biosynthesis.

The aldehyde pentaglutamate derivatives 21 and 25 and dimethylhydrazone pentaglutamate derivatives 22 and 26 exhibited little or no cytotoxic activity presumably due to difficulty in traversing the cellular membrane.

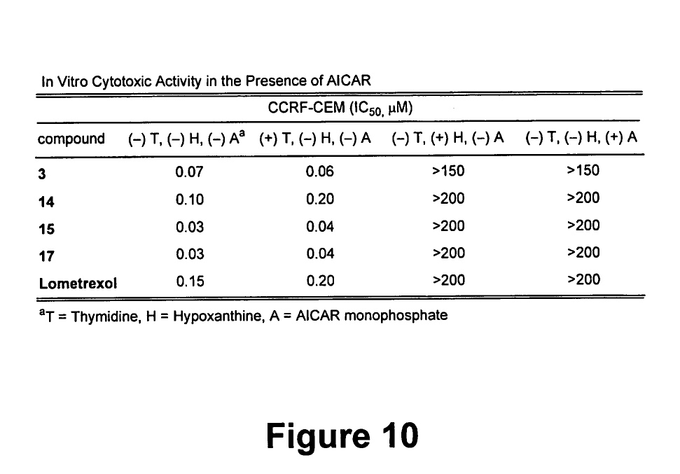

AICAR rescue experiments were performed using 3, 14, 15, and 17 in order to further elucidate the source of their cytotoxic activity (FIG. 10). In each case, the reversal or rescue of the cytotoxicity with hypoxanthine (100 mM) or AICAR monophosphate (100 mM) resulted in a ³10³-10⁴ increase in the IC₅₀ value. This indicates that the activity is being observed through selective inhibition of purine biosynthesis prior to the AICAR Tfase enzymatic step, presumably through inhibition of GAR Tfase. This selective sensitivity to GAR Tfase is the expected behavior of the inhibitors 14, 15, and 17, whereas the aldehyde 3 and the corresponding γ-pentaglutamates 21 (from 3) and 22 (from 15) would be expected to be more effective or at least as effective at acting on AICAR Tfase.

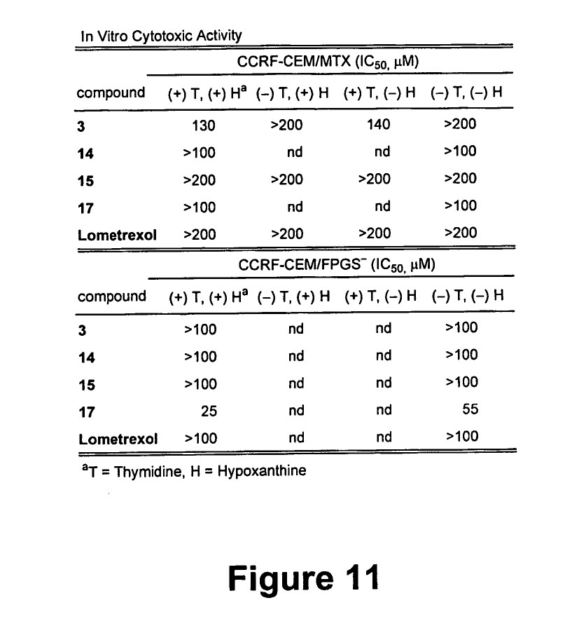

The extent to which the potent cytotoxic activity of 3, 14, 15, and 17 was dependent on reduced folate transport across the cellular membrane was established by assaying against a mutant CCRF-CEM cell line (CEM/MTX) (FIG. 11). This cell line has been shown to have an impaired reduced folate carrier (Jansen, G., et al., Cancer Res. 1989, 49, 2455). All the potent inhibitors including 3 and 15 lost cytotoxic activity against this mutant CCRF-CEM/MTX cell line (IC₅₀>100 mM), indicating that reduced folate carrier transport is essential for their biological activity.

Finally, the extent to which the potent cytotoxic compounds were dependent upon polyglutamation was established by assaying against a mutant CCRF-CEM cell line (CEM/FPGS⁻) that lacks folylpolyglutamate synthase (FPGS) (The CCRF-CEM/FPGS⁻ cell line was kindly provided by Professor G. P. Beardsley (Yale)) (FIG. 11). All the potent inhibitors including 3 and 15 lost cytotoxic activity against this cell line indicating that inhibitor polyglutamation is essential for their biological activity.

Human GAR Tfase Inhibition. In the preceding studies, the four key inhibitors 3, 14, 15, and 17 exhibited exceptionally potent cytotoxic activity (e.g., 3 IC₅₀=60 nM) that proved sensitive to media purines and AICAR, that required reduced folate carrier transport into the cells, and that required polyglutamation. This level of functional potency surpassed the enzyme inhibition activity approximately 100-fold (Kᵢ=6 mM for 3 versus E. coli GAR Tfase) suggesting that the cytotoxic potency enhancement might rest with intracellular accumulation of the inhibitors. However, human AICAR Tfase was found to be more potently inhibited by the γ-pentaglutamates inconsistent with GAR Tfase being the target suggesting that intracellular accumulation by transport and polyglutamation might be only part of the answer. Consequently, the inhibitors were examined against recombinant human GAR Tfase (rhGAR Tfase) and a remarkable and unprecedented sensitivity to the aldehyde inhibitor 3 was observed.

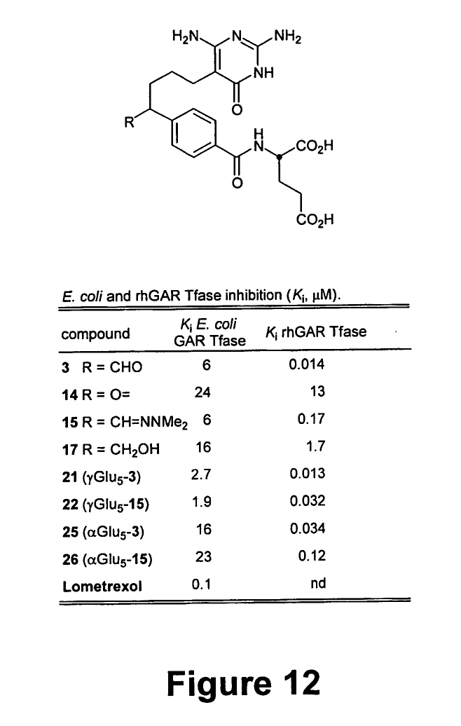

The results of the examination of 3, 14, 15, and 17 as well as the corresponding α- and γ-pentaglutamates of 3 and 15 (compounds 21, 22, 25 and 26) against recombinant human GAR Tfase are summarized in FIG. 12 alongside the results against E. coli GAR Tfase. Remarkably, the inhibitors displayed trends against rhGAR Tfase that might be expected of the original design. For the monoglutamates, the aldehyde 3 proved to be an exceptionally potent inhibitor of rhGAR Tfase (Kᵢ =14 nM) and it was approximately 10-fold more potent than the corresponding dimethylhydrazone 15 (Kᵢ=170 nM), 100-fold more potent than the corresponding alcohol 17 (Kᵢ=1.7 mM), and 1000-fold more potent than the degradation ketone 14 (Kᵢ =13 mM). The γ-pentaglutamates of 3 and 15 (21 and 22) were roughly 3-4′ more potent than the corresponding α-pentaglutamates 25 and 26. Whereas the γ-pentaglutamate of aldehyde 3 did not enhance its remarkable potency against rhGAR Tfase (3 versus 21), the γ-pentaglutamate of the hydrazone 15 did increase the potency 5-fold (15 versus 22). Most notably, this potency of the γ-pentaglutamates against rhGAR Tfase is comparable to the cytotoxic potencies observed with 3 and 15 and consistent with the studies indicating that their target is GAR Tfase and not AICAR Tfase.

This striking difference in the behavior of the E. coli versus human GAR Tfase toward the inhibitors represents the first such demonstration of an unexpectedly selective inhibition of the human enzyme. In total, there are 20 enzyme residues that constitute the core of the folate binding site and 12 enlist their side chains to stabilize folate binding. These 12 are identical in the human and E. coli enzymes except for one conservative Leu-143 (E. coli) versus Val-143 (human) substitution. This has led to the expectation that little inhibitor distinction between the E. coli and human enzymes might be observed. The results with 3, which is over 400-fold more potent against the human versus E. coli enzyme, indicate that this need not be the case.

Experimental

### Methyl 4-[1-Dimethylhydrazono-5-bromopent-2-yl]benzoate (7)

A solution of diisopropylamine (1.82 mL, 13.0 mmol, 1.5 equiv) in THF (13 mL) cooled to 0° C. was treated with n-BuLi (2.5 M in hexanes, 4.50 mL, 11.2 mmol, 1.3 equiv) and stirred at 0° C. for 30 min and at −78° C. for 20 min. A solution of 6 (1.91 g, 8.65 mmol, 1.0 equiv) in THF (3.9 mL) was added dropwise and the resulting solution was stirred at −78° C. for 30 min. A solution of 1,3-dibromopropane (8.79 mL, 86.5 mmol, 10.0 equiv) in HMPA (5.5 mL) was added and the mixture was stirred at −78° C. for 2 h. The reaction mixture was quenched by the dropwise addition of saturated aqueous NH₄Cl (10 mL) and allowed to warm to 25° C. The reaction mixture was diluted with EtOAc (200 mL) and washed successively with H₂O (2′ 50 mL) and saturated aqueous NaCl (50 mL). The organic layer was dried (Na₂SO₄), filtered, and concentrated under reduced pressure. Chromatography (SiO₂, 5′ 15 cm, 70% hexanes-EtOAc) provided 7 (1.55 g, 52%) as a yellow oil: MALDIFTMS (DHB) m/z 341.0856 (M+H⁺, C₁₅H₂₁BrN₂O₂ requires 341.0859).

### Methyl 4-[6-Carboethoxy-6-cyano-1-(dimethylhydrazono)hex-2-yl]benzoate (8)

A suspension of NaH (60% dispersion, 0.211 g, 5.28 mmol, 1.2 equiv) in anhydrous DMF (25 mL) at 0° C. was treated dropwise with ethyl cyanoacetate (0.61 mL, 5.7 mmol, 1.3 equiv). The solution was stirred at 0° C. for 30 min, forming the sodium salt as a clear solution. This solution was treated with a solution of 7 (1.50 g, 4.40 mmol) in anhydrous DMF (10 mL). The resulting reaction mixture was stirred at 25° C. for 2.5 h. The reaction mixture was diluted with EtOAc (200 mL) and washed successively with H₂O (3′ 50 mL) and saturated aqueous NaCl (50 mL). The organic layer was dried (Na₂SO₄), filtered, and concentrated under reduced pressure. Chromatography (SiO₂, 4′ 15 cm, 60% hexanes-EtOAc) provided 8 (0.798 g, 49%) as a yellow oil: MALDIFTMS (DHB) m/z 374.2066 (M+H⁺, C₂₀H₂₇N₃O₄ requires 374.2074).

### Methyl 4-[5-(2,4-Diamino-6(1H-pyrimidinon-5-yl)-1-(dimethylhydrazono)-pent-2-yl]benzoate (9)

A solution of Na metal (0.035 g, 1.54 mmol, 2.2 equiv) in CH₃OH (0.87 mL) at 25° C. was treated with guanidine hydrochloride (0.073 g, 0.769 mmol, 1.1 equiv). The solution was stirred at 25° C. for 30 min and then treated with a solution of 8 (0.261 mg, 0.698 mmol) in CH₃OH (0.87 mL). The solution was stirred at 25° C. for 12 h. The excess NaOCH₃ was neutralized by the addition of HOAc (0.045 mL). Chromatography (SiO₂, 3′ 15 cm, 10% CH₃OH—CHCl₃) provided 9 (0.140 g, 52%) as a white solid: MALDIFTMS (DHB) m/z 409.1963 (M+Na⁺, C₁₉H₂₆N₆O₃ requires 409.1964).

### 4-[5-(2,4-Diamino-6(1H-pyrimidinon-5-yl)-1-(dimethylhydrazono)pent-2-yl]benzoic Acid (10)

A solution of 9 (0.063 g, 0.163 mmol) in 3:1 CH₃OH—H₂O (1.63 mL) was treated with LiOH—H₂O (0.021 g, 0.489 mmol, 3.0 equiv) and the mixture was stirred at 25° C. for 12 h. The mixture was diluted with H₂O (10 mL) and the aqueous layer was washed with EtOAc (3′ 3 mL). The aqueous layer was acidified to pH=4 by the addition of 1 M aqueous HCl. The solution was concentrated under reduced pressure and the residue was treated with toluene (3′ 5 mL) to remove traces of H₂O to provide 10 (0.053 g, 88%): MALDIFTMS (DHB) m/z 395.1824 (M+Na⁺, C₁₈H₂₄N₆O₃ requires 395.1824).

### Di-tert-butyl N-(4-[5-(2,4-Diamino-6(1H-pyrimidinon-5-yl)-1-(dimethylhydrazono)pent-2-yl]benzoyl 1-L-glutamate (11)

A solution of 10 (0.029 g, 0.078 mmol) and di-tert-butyl L-glutamate hydrochloride (0.034 g, 0.117 mmol, 1.5 equiv) in DMF (0.31 mL) was treated with NaHCO₃ (0.020 g, 0.234 mmol, 3.0 equiv) followed by EDCI (0.045 g, 0.234 mmol, 3.0 equiv). The reaction mixture was stirred at 25° C. for 12 h before the solvent was removed under reduced pressure. The residue was dissolved in CHCl₃ (5 mL) and extracted with saturated aqueous NaHCO₃ (2 mL). The organic layer was dried (Na₂SO₄), filtered, and concentrated under reduced pressure. PCTLC (SiO₂, 2 mm plate, 10% CH₃OH—CHCl₃) provided 11 (0.017 g, 36%) as a white solid: MALDIFTMS (DHB) m/z 614.3678 (M+H⁺, C₃₁H₄₇N₇O₆ requires 614.3666).

### Di-tert-butyl N-{4-[4-(2,4-Diamino-6(1H)-pyrimidinon-5-yl)-1-formyl-but-2-yl]benzoyl}-L-glutamate (12)

A solution of 11 (30 mg, 0.049 mmol) in THF (0.9 mL) and pH 7 aqueous phosphate buffer (0.02 mL) cooled to 0° C. was treated with a solution of CuCl₂ (33 mg, 0.244 mmol, 5.0 equiv) in H₂O (0.3 mL). The solution was stirred at 0° C. for 1 h before it was quenched by the dropwise addition of a pH 8 saturated aqueous NH₄Cl—NH₄OH solution (20 mL). The solution was extracted with CHCl₃ (3′ 20 mL), purged with N₂, dried (Na₂SO₄), filtered, and concentrated under reduced pressure. PCTLC (SiO₂, 1 mm plate, 20% CH₃OH—CHCl₃) provided 12 (11 mg, 39%; typically 21-44%) as a white solid: MALDIFTMS (DHB) m/z 594.2904 (M+Na⁺, C₂₉H₄₁N₅O₇ requires 594.2904).

### Di-tert-butyl N-{4-[4-(2,4-Diamino-6(1H-pyrimidinon-5-yl)-1-oxo-but-1-yl]benzoyl}-L-glutamate (13)

Obtained as the higher R_{f} spot from the reaction that provided 12. PCTLC (SiO₂, 1 mm plate, 20% CH₃OH—CHCl₃) provided 13 (12 mg, 44%) as a white solid: MALDIFTMS (DHB) m/z 580.2730 (M+Na⁺, C₂₈H₃₉N₅O₇ requires 580.2747).

### N-{4-[4-(2,4-Diamino-6(1H-pyrimidinon-5-yl)-1-formyl-but-2-yl]benzoyl}-L-glutamic Acid (3)

A solution of 12 (2.9 mg, 0.0051 mmol) in CHCl₃ (0.20 mL) cooled to 0° C. was treated with trifluoroacetic acid (0.04 mL). The solution was stirred at 0° C. for 2 h and 25° C. for 12 h. Et₂O (1 mL) was added and a precipitate formed. The precipitate was triturated with Et₂O (3′ 1 mL) and dried in vacuo to give 3-CF₃CO₂H (2.6 mg, 89%) as a tan solid: MALDIFTMS (DHB) m/z 482.1652 (M+Na⁺, C₂₁H₂₅N₅O₇ requires 482.1670).

### N-{4-[4-(2,4-Diamino-6(1H)-pyrimidinon-5-yl)-1-oxo-but-1-yl]benzoyl}-L-glutamic Acid (14)

A solution of 13 (3.3 mg, 0.0059 mmol) in CHCl₃ (0.12 mL) cooled to 0° C. was treated with trifluoroacetic acid (0.01 mL). The solution was stirred at 0° C. for 2 h and 25° C. for 12 h. Et₂O (1 mL) was added and a precipitate formed. The precipitate was triturated with Et₂O (3′ 1 mL) and dried in vacuo to give 14-CF₃CO₂H (2.7 mg, 83%) as a white solid: MALDIFTMS (DHB) m/z 446.1674 (M+H⁺, C₂₀H₂₃N₅O₇ requires 446.1676).

### N{4-[5-(2,4-Diamino-6(1H)-pyrimidinon-5-yl)-1-(dimethylhydrazono)pent-2-yl]benzoyl}-L-glutamic Acid (15)

A solution of 11 (7.5 mg, 0.0122 mmol) in CHCl₃ (0.20 mL) cooled to 0° C. was treated with trifluoroacetic acid (0.05 mL). The solution was stirred at 0° C. for 2 h and 25° C. for 12 h. The reaction was concentrated under reduced pressure. The product was triturated with Et₂O (1′ 1 mL) and dried in vacuo to give 15-CF₃CO₂H (7.5 mg, 100%) as a white solid: MALDIFTMS (DHB) m/z 524.2248 (M+Na⁺, C₂₃H₃₁N₇O₆ requires 524.2233).

### Di-tert-butyl N{4-[5-(2,4-Diamino-6(1H-pyrimidinon-5-yl)-1-hydroxypent-2-yl]benzoyl}-L-glutamate (16)

A solution of 12 (6.1 mg, 0.0107 mmol) in CH₃OH (0.11 mL) at 0° C. was treated with NaBH₄ (1.2 mg, 0.032 mmol, 3.0 equiv). The solution was stirred at 0° C. for 2 h and 25° C. for 2 h before the solvent was removed under reduced pressure. The residue was diluted with CHCl₃ (2 mL) and washed successively with saturated aqueous NH₄Cl (1 mL) and saturated aqueous NaCl (1 mL), dried (Na₂SO₄), filtered, and concentrated under reduced pressure. PCTLC (SiO₂, 1 mm plate, 8% CH₃OH—CHCl₃) provided 16 (5.4 mg, 88%) as a white solid: MALDIFTMS (DHB) m/z 574.3263 (M+H⁺, C₂₉H₄₃N₅O₇ requires 574.3241).

### N-{4-[5-(2,4-Diamino-6(1H)-pyrimidinon-5-yl)-1-hydroxypent-2-yl]benzoyl}-L-glutamic Acid (17)

A solution of 16 (4.2 mg, 0.0073 mmol) in CHCl₃ (0.20 mL) cooled to 0° C. was treated with trifluoroacetic acid (0.02 mL). The solution was stirred at 0° C. for 2 h and 25° C. for 12 h. Et₂O (1 mL) was added and a precipitate formed. The precipitate was triturated with Et₂O (3′ 1 mL) and dried in vacuo to give 17-CF₃CO₂H (4.1 mg, 98%) as a white solid: MALDIFTMS (DHB) m/z 484.1824 (M+Na⁺, C₂₁H₂₇N₅O₇ requires 484.1808).

### N-{4-[5-(2,4-Diamino-6(1H)-pyrimidinon-5-yl)-1-(dimethylhydrazono)pent-2-yl]benzoyl}-L-γ-glutamyl-L-γ-glutamyl-L-γ-glutamyl-L-γ-glutamyl-L-γ-glutamic Acid Hexa-tert-butyl Ester (19)

A solution of 10 (90 mg, 0.24 mmol) and 18⁴⁴ (242 mg, 0.24 mmol, 1.0 equiv) in anhydrous DMF (1.0 mL) was treated with NaHCO₃ (61 mg, 0.73 mmol, 3.0 equiv) followed by EDCI (139 mg, 0.73 mmol, 3.0 equiv) and stirred at 25° C. for 12 h. The reaction mixture was diluted with EtOAc (50 mL) and washed with saturated aqueous NaHCO₃ (2′ 20 mL) followed by saturated aqueous NaCl (20 mL). The organic layer was dried (Na₂SO₄), filtered, and concentrated under reduced pressure. Chromatography (SiO₂, 3′ 15 cm, 10% CH₃OH—CHCl₃) provided 19 (102 mg, 31%) as a white solid: MALDIFTMS (DHB) m/z 1354.7844 (M+H⁺, C₆₇H₁₀₇N₁₁O₁₈ requires 1354.7868).

### N-{4-[4-(2,4-Diamino-6(1H-pyrimidinon-5-yl)-1-formyl-but-2-yl]benzoyl}-L-γ-glutamyl-L-γ-glutamyl-L-γ-glutamyl-L-γ-glutamyl-L-γ-glutamic Acid Hexa-tert-butyl Ester (20)

A solution of 19 (41 mg, 0.030 mmol) in THF (0.43 mL) and pH 7 aqueous phosphate buffer (0.09 mL) cooled to 0° C. was treated with a solution of CuCl₂ (20.4 mg, 0.15 mmol, 5.0 equiv) in H₂O (0.15 mL). The solution was stirred at 0° C. for 1.5 h before it was quenched by the dropwise addition of a pH 8 saturated aqueous NH₄C₁—NH₄OH solution (5 mL). The solution was extracted with CHCl₃ (3′ 10 mL), purged with N₂, dried (Na₂SO₄), filtered, and concentrated under reduced pressure. PCTLC (SiO₂, 1 mm plate, 8% CH₃OH—CHCl₃) provided 20 (19 mg, 48%) as a white solid: MALDIFTMS (DHB) m/z 1312.7312 (M+H⁺, C₆₅H₁₀₁N₉O₁₉ requires 1312.7286).

### N{4-[5-(2,4-Diamino-6(1H)-pyrimidinon-5-yl)-1-(dimethylhydrazono)pent-2-yl]benzoyl}-L-γ-glutamyl-L-γ-glutamyl-L-γ-glutamyl-L-γ-glutamyl-L-γ-glutamic Acid (22)

A solution of 19 (25 mg, 0.019 mmol) in CHCl₃ (1.00 mL) cooled to 0° C. was treated with trifluoroacetic acid (0.25 mL). The solution was stirred at 0° C. for 2 h and 25° C. for 12 h. The solution was concentrated under reduced pressure. The solid residue was triturated with Et₂O (3′ 5 mL) and dried in vacuo to give 22-CF₃CO₂H (21 mg, 100%) as a tan solid: MALDIFTMS (DHB) m/z 1018.4102 (M+H⁺, C₄₃H₅₉N₁₁O₁₈ requires 1018.4112).

### N-{4-[5-(2,4-Diamino-6(1H)-pyrimidinon-5-yl)-1-formyl-but-2-yl]benzoyl}-L-γ-glutamyl-L-γ-glutamyl-L-γ-glutamyl-L-γ-glutamyl-L-γ-glutamic Acid (21)

A solution of 20 (16 mg, 0.012 mmol) in CHCl₃ (1.00 mL) cooled to 0° C. was treated with trifluoroacetic acid (0.25 mL). The solution was stirred at 0° C. for 2 h and 25° C. for 12 h. The solution was concentrated under reduced pressure. The solid residue was triturated with Et₂O (3′ 5 mL) and dried in vacuo to give 21-CF₃CO₂H (13 mg, 100%) as a tan solid: MALDIFTMS (DHB) m/z 976.3570 (M+H⁺, C₄₁H₅₃N₉O₁₉, requires 976.3530).

### N-{4-[5-(2,4-Diamino-6(1H)-pyrimidinon-5-yl)-1-(dimethylhydrazono)pent-2-yl]benzoyl}-L-α-glutamyl-L-α-glutamyl-L-α-glutamyl-L-α-glutamyl-L-α-glutamic Acid Hexa-tert-butyl Ester (24)

A solution of 10 (7.5 mg, 0.020 mmol) and 23⁴⁵ (20.2 mg, 0.020 mmol, 1.0 equiv) in DMF (0.1 mL) was treated with NaHCO₃ (5.1 mg, 0.060 mmol, 3.0 equiv) followed by EDCI (11.6 mg, 0.060 mmol, 3.0 equiv) and stirred at 25° C. for 48 h. The reaction was diluted with EtOAc (20 mL) and washed with saturated aqueous NaHCO₃ (5 mL). The organic layer was dried (Na₂SO₄), filtered, and concentrated under reduced pressure. PCTLC (SiO₂, 2 mm plate, 10% CH₃OH—CHCl₃) provided 24 (6.0 mg, 22%) as a white solid: MALDIFTMS (DHB) m/z 1376.7730 (M+Na⁺, C₆₇H₁₀₇N₁₁O₁₈ requires 1376.7687).

### N{4-[5-(2,4-Diamino-6(1H)-pyrimidinon-5-yl)-1-(dimethylhydrazono)pent-2-yl]benzoyl}-L-α-glutamyl-L-α-glutamyl-L-α-glutamyl-L-α-glutamyl-L-α-glutamic Acid (25)

A solution of 24 (19 mg, 0.014 mmol) in THF (0.2 mL) and pH 7 aqueous phosphate buffer (0.04 mL) cooled to 0° C. was treated with a solution of CuCl₂ (9.4 mg, 0.070 mmol, 5.0 equiv) in H₂O (0.07 mL). The solution was stirred at 0° C. for 1 h before it was quenched by the dropwise addition of a pH 8 saturated aqueous NH₄C₁—NH₄OH solution (5 mL). The solution was extracted with CHCl₃ (3′ 5 mL), purged with N₂, dried (Na₂SO₄), filtered, and concentrated under reduced pressure. PCTLC (SiO₂, 1 mm plate, 8% CH₃OH—CHCl₃) removed baseline impurities. The isolated product was dissolved in CHCl₃ (1.00 mL), cooled to 0° C. and treated with trifluoroacetic acid (0.25 mL). The solution was stirred at 0° C. for 2 h and 25° C. for 12 h. The solution was concentrated under reduced pressure. The solid residue was triturated with Et₂O (3′ 5 mL) and dried in vacuo to give 25-CF₃CO₂H (3.4 mg, 22% over two steps from 24) as a tan solid: MALDIFTMS (DHB) m/z 976.3517 (M+H⁺, C₄₁H₅₃N₉O₁₉ requires 976.3530).

### N{4-[5-(2,4-Diamino-6(1H)-pyrimidinon-5-yl)-1-formyl-but-2-yl]benzoyl}-L-α-glutamyl-L-α-glutamyl-L-α-glutamyl-L-α-glutamyl-L-α-glutamic Acid (26)

A solution of 24 (5.4 mg, 0.0040 mmol) in CHCl₃ (1.00 mL) cooled to 0° C. was treated with trifluoroacetic acid (0.25 mL). The solution was stirred at 0° C. for 2 h and 25° C. for 12 h. The solution was concentrated under reduced pressure. The solid residue was triturated with Et₂O (3′ 5 mL) and dried in vacuo to give 26-CF₃CO₂H (4.5 mg, 100%) as a tan solid: MALDIFTMS (DHB) m/z 1018.4157 (M+H⁺, C₄₃H₅₉N₁₁O₁₈ requires 1018.4112).

GAR Tfase, AICAR Tfase, and DHFR Inhibition.

GAR and AICAR Tfase inhibition studies were conducted as previously detailed²⁸ with the exception that the AICAR Tfase inhibition was conducted in the absence of 5 mM β-mercaptoethanol and screened with 10 nM enzyme, 25 mM inhibitor and 22.5 mM of cofactor. The DHFR inhibition study was conducted as previously detailed with 10 nM enzyme, 30 mM H₂F, 100 mM NADPH and 30 mM inhibitor.

### Design of Advanced Inhibitors

It has been previously disclosed that folate-based inhibitors that incorporate electrophilic functional groups that could potentially interact either with active site nucleophiles or with the GAR/AICAR substrate amine (Boger, D. L., et al., Bioorg. Med. Chem. 1997, 5, 1839-1846; Boger, D. L., et al., Bioorg. Med. Chem. 1998, 6, 643-659; Boger, D. L., et al., Bioorg. Med. Chem. Lett. 2000, 10, 1471-1475; Boger, D. L., et al., Bioorg. Med. Chem. 2000, 8, 1075-1086; Boger, D. L., et al., Bioorg. Med. Chem. 1997, 5, 1817-1830; and Boger, D. L., et al., Bioorg. Med. Chem. 1997, 5, 1847-1852). The most significant of these was the folate-based inhibitor 10-formyl-TDAF (103) (Boger, D. L., et al., Bioorg. Med. Chem. 1997, 5, 1817-1830). X-ray and NMR studies of the inhibitor-enzyme complexes revealed that the inhibitors bound as their gem-diols (Greasley, S. E., et al., Biochemistry 1999, 38, 16783-16793). The formation of the gem-diol mimics the formyl transfer tetrahedral intermediate and provides strong hydrogen bond interactions between the inhibitor and protein.

Even though 10-formyl-TDAF (103) was a relatively potent GAR Tfase inhibitor (Kᵢ=260 nM), it failed to exhibit effective cytotoxic activity attributable to a combination of properties including poor stability, ineffective transport by the reduced folate carrier, and inefficient intracellular polyglutamation by FPGS (Boger, D. L., et al., Bioorg. Med. Chem. 1997, 5, 1817-1830). In contrast, 10-formyl-DDACTHF (3), which replaces the quinazoline of 103 with a diaminopyrimidinone, is not only an effective GAR Tfase inhibitor, but a potent cytotoxic agent (CCRF-CEM IC₅₀=60 nM). Effective transport by the reduced folate carrier and efficient polyglutamation by FPGS were found to contribute to the cytotoxic activity by enhancing its intracellular accumulation. Moreover, 10-formyl-DDACTHF (3) proved to be remarkably selective for human GAR Tfase (Kᵢ=14 nM against rhGAR Tfase) compared to E. coli GAR Tfase (Kᵢ=6 μM). Nevertheless, a facile oxidative decarbonylation of the key formyl group conveyed a chemical instability to 3, detracting from its in vivo utility.

The use of trifluoromethyl ketones as reversible enzyme inhibitors has seen wide application, most notably in the field of serine proteases (Wolfenden, R., Annu. Rev. Biophys. Bioeng. 1976, 5, 271-306; Brodbeck, U., et al., Biochim. Biophys. Acta 1979, 567, 357-369; and Gelb, M. H., et al., Biochemistry 1985, 24, 1813-1817). Herein, a trifluoromethyl ketone was introduced to replace the aldehyde of compound 3. The trifluoromethyl ketone can serve to stabilize gem-diol formation of the electrophilic carbonyl to a greater extent than a formyl group and, hence, can promote active site binding by mimicking the tetrahedral intermediate of the formyl transfer reaction. By replacing N10 with a carbon, the inhibitor precludes formyl transfer yet can competitively bind to the folate binding site (Boger, D. L., et al., Bioorg. Med. Chem. 1997, 5, 1817-1830). Such inhibitors display an enhanced affinity for folate-dependent enzymes involved in formyl transfer reactions, e.g. GAR Tfase and AICAR Tfase, and, therefore, exhibit selectivity towards these enzymes versus those involved in methyl or methylene transfer, such as thymidylate synthetase (TS).

Like 3, analogue 101 is transported into cells by the reduced folate carrier and to be a substrate for FPGS effectively sequestering it. It was also envisioned that 101 may exhibit enhanced chemical stability and pharmacological properties in comparison to aldehyde 3. Thus, the evolution of compound 101 required assessment of a number of factors which include improved stability, the ability to enter cells by pathways involving the reduced folate carrier or folate-binding membrane protein transport system, efficient conversion within the cell to polyglutamated forms by FPGS, and optimization of the selectivity and affinity of the inhibitor for its target enzyme.

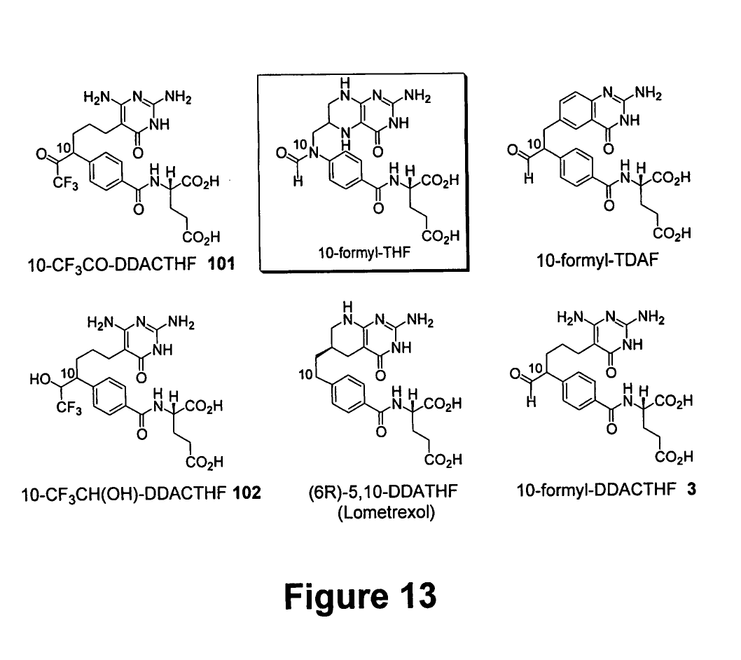

Herein, the knowledge obtained from various X-ray structures of GAR Tfase has been applied to design an advanced compound that acts as a tight binding, specific inhibitor of GAR Tfase. A previous structure of E. coli GAR Tfase (PDB code 1C2T) in complex with substrate and 10-formyl-TDAF (103, FIG. 13), a cofactor analogue bearing a nontransferable formyl group (E. coli GAR Tfase Kᵢ=260 nM), revealed that the inhibitor bound as the hydrated aldehyde (gem-diol) in the enzyme active site (Greasley, S. E., et al., Biochemistry 1999, 38, 16783-16793), mimicking the formyl transfer tetrahedral intermediate.

As a result of the synthesis and evaluation of an extensive series of related candidate inhibitors, mainly based on the E. coli GAR Tfase complex structures, the design, synthesis, and evaluation of an advanced folate analogue, 10-CF₃CO-DDACTHF (101) (FIG. 13) is disclosed herein. This compound exhibits selective and tight binding affinity towards human GAR Tfase (Kᵢ=15 nM), and is at least 10 times more potent than Lometrexol in cytotoxic assays. The analogue also exhibits excellent stability and solubility at pH 7-8, and possesses a number of additional properties that make it an excellent candidate for in vivo antitumor testing in animal models. The crystal structure of human GAR Tfase with the inhibitor at 1.98 Å resolution at physiological pH defines the detailed interactions and geometry of the inhibitor within the active binding site, and provides the best model to date for computational docking studies to explore the mechanism of binding of the natural folate cofactor. Docking of 10-formyl-THF (FIG. 13) into the binding site strongly supports the conclusions from the kinetic and mutagenesis data for the central role played by the catalytic triad (His108, Asp144 and Asn106), and provides a more physiologically relevant model on which to base future inhibitor designs.

Inhibitor Synthesis and Chemical Characterization.

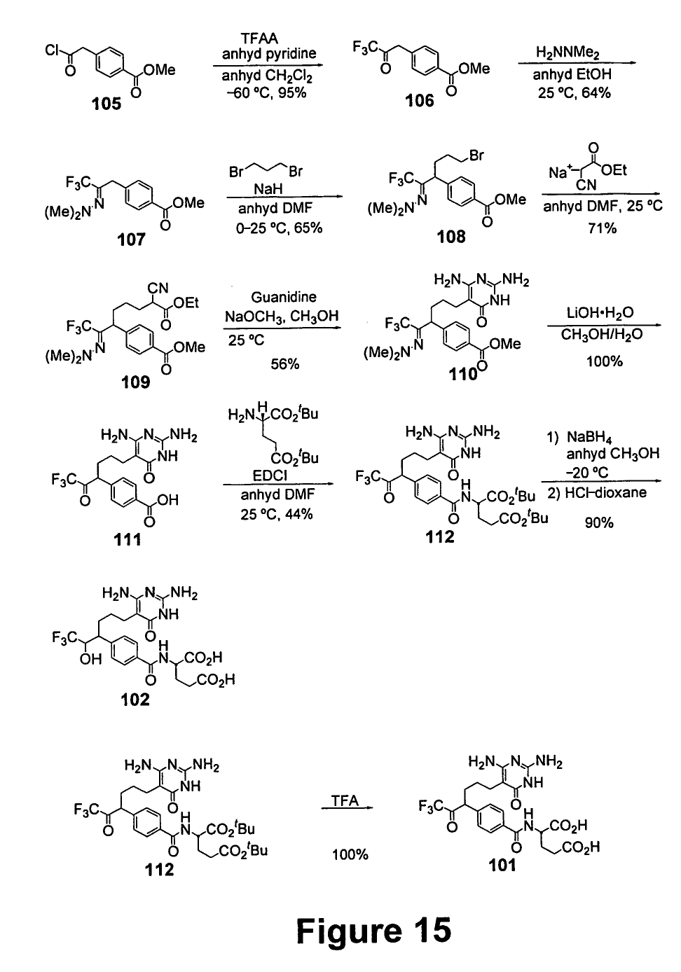

The synthesis of 101 was accomplished as outlined in FIG. 15. The known acid chloride 105 (Arakawa, K., et al., Chem. Pharm. Bull. (Tokyo) 1997, 45, 1984-1993) was converted to the corresponding trifluoromethyl ketone 106 by reaction of trifluoroacetic anhydride (pyridine, CH₂Cl₂, −60° C., 4 h) followed by aqueous quench (95%) (Biovin, J., et al., Tetrahedron Lett. 1992, 33, 1285-1288; and Biovin, J., et al., Tetrahedron 1995, 51, 2573-2584). The ¹H NMR (CD₃OD) spectra of 106 clearly indicated peaks corresponding to the benzylic methylene protons at δ3.34 and 3.1, as well as the absence of a peak corresponding to an enol methine, indicating that this compound exists as a hemiketal in CD₃OD, which was further corroborated by ¹³C NMR (CD₃OD). Reaction of 106 with N,N-dimethylhydrazine (glacial AcOH, anhydrous EtOH, 25° C., 48 h, 64%) provided the key N,N-dimethylhydrazone 107. NaH deprotonation of 107 (DMF, 0° C., 15 min) and subsequent treatment with excess 1,3-dibromopropane (6 equiv, DMF, 25° C., 2.5 h, 65%) provided the monoalkylation product 108. The pre-formed sodium salt of ethyl cyanoacetate (NaH, DMF, 0° C., 30 min) was alkylated with 108 (DMF, 25° C., 2 h) to give 109 (71%), and treatment with the free base of guanidine (1.2 equiv, CH₃OH, 25° C., 1 h) under basic conditions gave the desired pyrimidinone 110 (FIG. 15). Treatment of 110 with LiOH (3 equiv, 3:1 CH₃OH—H₂O, 25° C., 24 h) cleanly hydrolyzed both the methyl ester and the dimethylhydrazone, providing 111, which was coupled with di-tert-butyl L-glutamate hydrochloride (EDCI, NaHCO₃, DMF, 25° C., 72 h) to provide 112. Deprotection of 112 was accomplished by treatment with trifluoroacetic acid (1:4 v/v TFA/CHCl₃, 25° C., 12 h, 100%) to provide 10-CF₃CO-DDACTHF (101).

Most significantly, and unlike 103 and 3 which both suffer a facile oxidative decarbonylation reaction of the key formyl group (Boger, D. L., et al., Bioorg. Med. Chem. 1997, 5, 1817-1830), 101 was stable in pH 7-8 buffers in the presence of air showing no decomposition or reaction after 7 days. When 10-CF₃CO-DDACTHF (101) was characterized by ¹H and ¹³C NMR in CD₃OD, no ketone or enol form was observed, and 101 was found to be exclusively in the hemiacetal form. While this behavior is presumably solvent dependent, these studies indicate that 101 likely exists in the hydrate form (gem-diol) in aqueous buffer and under the assay conditions. Finally, no evidence of separable C10 diastereomers was observed with 101, indicating that the two diastereomers are in rapid equilibrium.

GAR Tfase Inhibition.

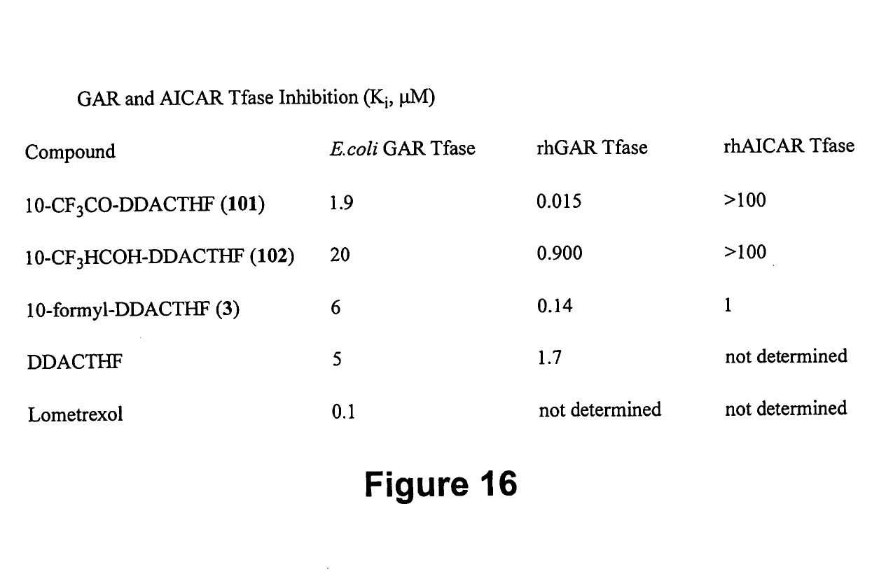

10-CF₃CO-DDACTHF (101) was assayed for inhibition of GAR Tfase and AICAR Tfase (FIG. 16) in comparison with other closely related folate-based GAR Tfase inhibitors. Compound 101 is a very effective inhibitor of rhGAR Tfase with a Kᵢ of 15 nM. Significantly, the compound shows selectivity towards the human enzyme rather than the E. coli enzyme to which it is 100-fold less potent with a Kᵢ of 1.9 μM, consistent with past observations with rhGAR and E. coli GAR Tfase. 101 (Kᵢ=15 nM) proved to be roughly 60-fold more potent than the corresponding alcohol 102 (Kᵢ=900 nM) and 100-fold more potent than DDACTHF (Kᵢ=1.7 μM) lacking a C10 substituent altogether, indicating that each of the gem-diol alcohols contributes to active site binding. Inhibition was not tested for the trifunctional enzyme, but previous data have indicated that the recombinant human GAR Tfase has comparable activity to the intact human trifunctional enzyme (Poch, M. T., et al., Protein Expr. Purif. 1998, 12, 17-24; Zhang, Y., et al., Biochemistry 2002, 41, 14206-14215; and Sanghani, S. P., et al., Biochemistry 1997, 36, 10506-10516).

10-CF₃CO-DDACTHF (101), as well as its corresponding alcohol 102, is a specific inhibitor for GAR Tfase, but is inactive (Kᵢ >100 μM) against other folate-dependent enzymes, including AICAR Tfase, DHFR and thymidylate synthetase (TS). These observations are consistent with the data below that show 101 derives its potent cytotoxic activity through inhibition of the purine, not pyrimidine, biosynthesis and at a step preceding the action of AICAR Tfase.

Biological Activity.

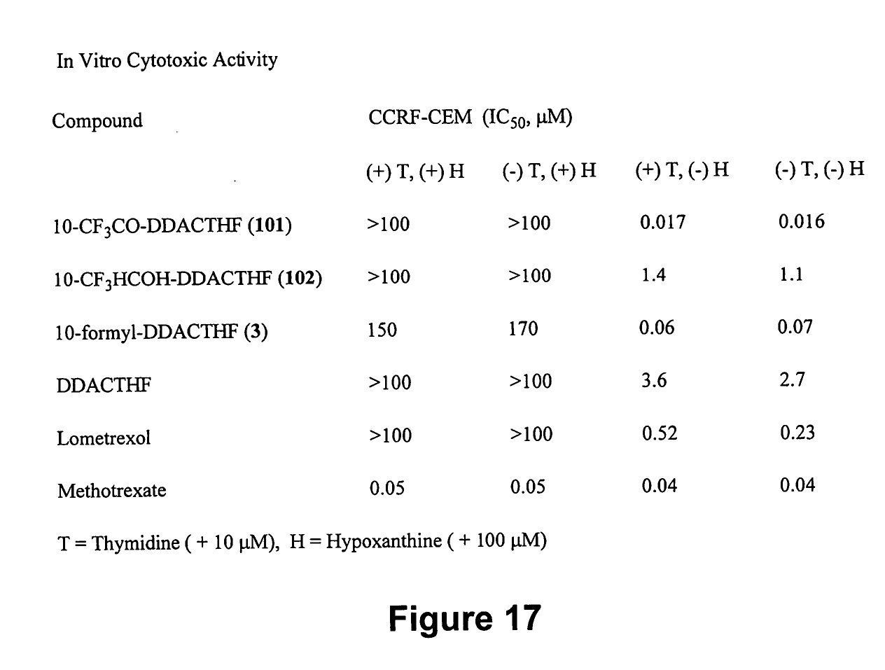

10-CF₃CO-DDACTHF (101) and its corresponding alcohol 2 were examined for CCRF-CEM cytotoxic activity in the presence (+) and absence (−) of added hypoxanthine (purine) or thymidine (pyrimidine) (FIG. 17). Compound 101 exhibits potent cytotoxic activity (IC₅₀=16 nM) against the CCRF-CEM cell line when purines (hypoxanthine) are absent in the media. Moreover, it is ca. 14-fold more potent than Lometrexol (IC₅₀=230 nM) and was inactive (IC₅₀>100 μM) in the presence of media purines. This sensitivity to the presence of purines, but not pyrimidines (thymidine), indicates that the cytotoxic activity of 101 is derived from inhibition of enzymes in the de novo purine biosynthetic pathway. This places 101 among the most potent, if not the most potent, inhibitor of human GAR Tfase yet disclosed. The related alcohol 102 (IC₅₀=1.1 μM) and DDACTHF lacking a C10 substituent (IC₅₀=2.7 μM) also exhibited cytotoxic activity, that was also sensitive to the presence of media purines. However, both are much less potent than ketone 101.

AICAR rescue experiments were also performed with 101 and its corresponding alcohol 102 in order to further define the source of their cytotoxic activity. In each case, the reversal or rescue of the cytotoxicity with hypoxanthine (100 μM) or AICAR monophosphate (100 μM) resulted in a 10³-10⁴ increase in the IC₅₀ (data not shown). Thus, the observed activity is due to selective inhibition of purine biosynthesis prior to the AICAR Tfase enzymatic step, consistent with inhibition of GAR Tfase. This selective sensitivity to GAR Tfase is the expected behavior of the inhibitors 101 and 102 based upon their inactivity against AICAR Tfase in vitro.

The extent to which the cytotoxic activity of 101 and 102 was dependent on reduced folate transport across the cellular membrane was established by assaying against a mutant CCRF-CEM cell line (CEM/MTX) (Sanghani, S. P., et al., Biochemistry 1997, 36, 10506-10516) which is deficient in the reduced folate carrier. Like Lometrexol, 101 and the related hydroxyl compound 102 lost activity against CCRF-CEM/MTX (data not shown) indicating the reduced folate carrier is required for activity and implying that they are effective substrates for transport.

Similarly, the importance of FPGS polyglutamation to cytotoxic activity was established by examining the inhibitor against a CCRF-CEM cell line deficient in FPGS (CCRF-CEM/FPGS⁻). Like Lometrexol, 101 and the related hydroxyl compound 102 (to a lesser extent) lacked or lost activity against this cell line (data not shown), indicating polyglutamation is required for activity of the inhibitors, presumably either by directly enhancing enzyme inhibitory activity, and/or as a consequence of intracellular accumulation of the inhibitors by preventing diffusion out of the cell.

X-Ray Structure Determination.

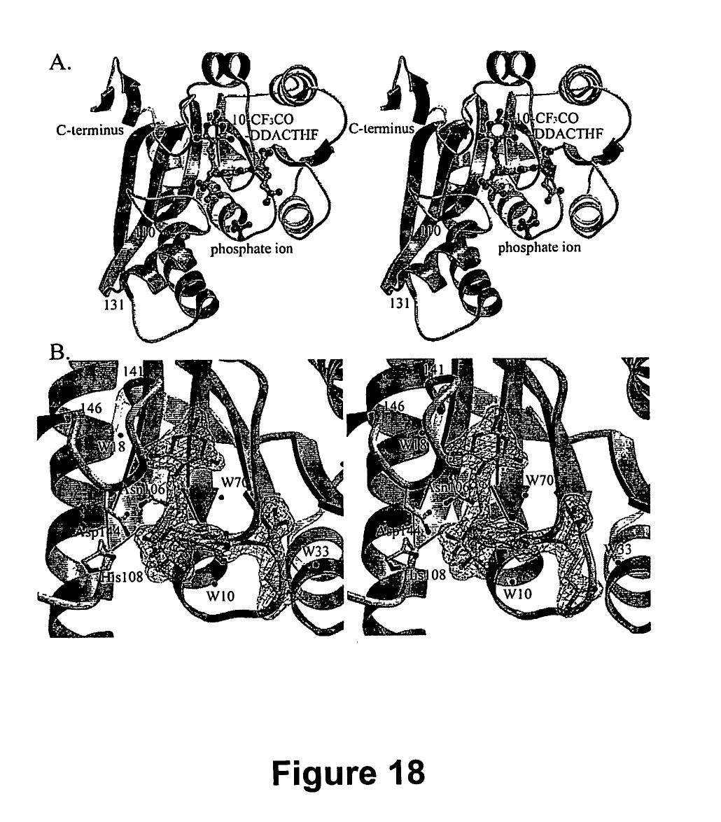

The crystal structure of rhGAR Tfase, cocrystallized with 101 at physiological pH 7, was determined at 1.98 Å resolution by MR using the unliganded human GAR Tfase at pH 8.5 (PDB code 1 MEJ) as the search model (FIG. 18). The crystal spacegroup is P3₁21 with two molecules per asymmetric unit, but no dimeric interaction is observed, consistent with other structures of human GAR Tfase, in which the enzyme always crystallizes as a monomer. The two monomers have very similar structures (main chain RMSD of 0.4 Å), and each contains a bound inhibitor 101 in the folate-binding site (FIG. 18). The final model of the complex includes residues 808-1007 from the trifunctional protein, with the last three residues not interpretable due to disorder. The numbering of the residues is the same as that for unliganded human GAR Tfase (Zhang, Y., et al., Biochemistry 2002, 41, 14206-14215).

The extent to which the cytotoxic activity of 101 and 102 was dependent on reduced folate transport across the cellular membrane was established by assaying against a mutant CCRF-CEM cell line (CEM/MTX) (Jansen, G., et al., Cancer Res. 1989, 49, 1959-1963) which is deficient in the reduced folate carrier. Like Lometrexol, 101 and the related hydroxyl compound 102 lost activity against CCRF-CEM/MTX (data not shown) indicating the reduced folate carrier is required for activity and implying that they are effective substrates for transport.

A preliminary 1.8 Å resolution structure has also been obtained for human GAR Tfase bound to 10-CF₃CO-DDACTHF (101) at pH 5. The only major difference is the previously observed conformational isomerism in the substrate binding pocket, in which the pocket is not accessible to the substrate at pH 5, but is open at pH 7 (Zhang, Y., et al., Biochemistry 2002, 41, 14206-14215). The folate-binding site is identical in the two structures (main chain RMSD of the folate-binding loop 140-146 is 0.08 Å).

Overall Structure.

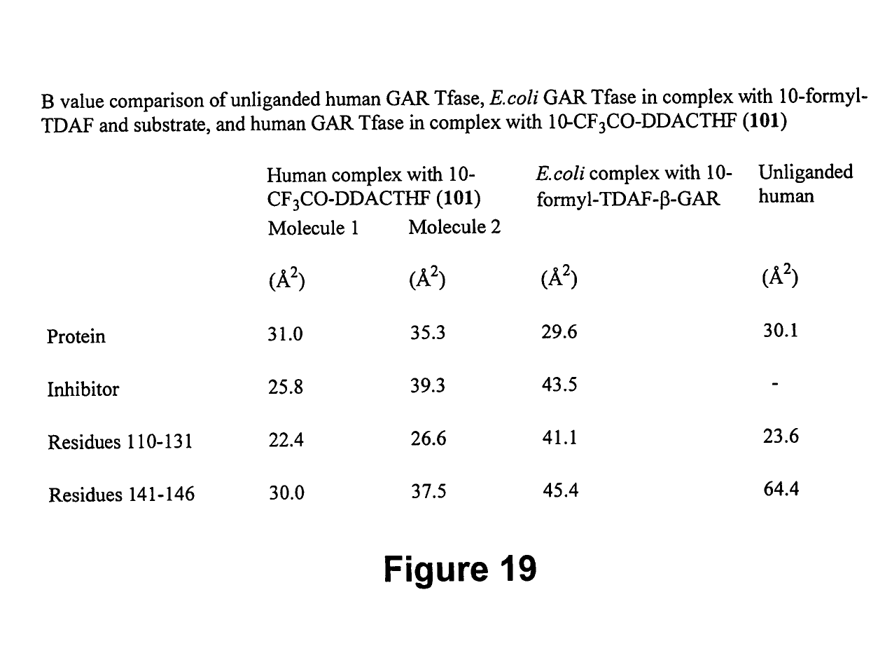

The overall topology of the complex between human GAR Tfase and 10-CF₃CO-DDACTHF (101) is very similar to the unliganded protein structure at pH 8.5 (PDB code 1 MEJ) (FIG. 18) (RMSD of 0.86 Å and 0.89 Å for molecules A and B). Molecule B has slightly higher thermal factors (average B of 35.3 Å²) than molecule A (average B of 31.0 A²) (FIG. 19). The loop helix 110-131 is highly ordered in the complex structure (B value of 24.5 Å²), consistent with the previous result that this loop-helix maintains a uniform conformation in human GAR Tfase (Zhang, Y., et al., Biochemistry 2002, 41, 14206-14215), unlike the pH-dependent order-disorder transition in the E. coli enzyme (FIG. 19). As substrate β-GAR was not present in the crystallization screens, the substrate-binding site was occupied by an inorganic phosphate ion (FIG. 18). Unlike the E. coli complex structures and unliganded human GAR Tfase in which the folate-binding loop 141-146 has very high B values, the same loop in the human enzyme has comparable B values (33.8 Å²) to the overall structure (33.0 Å²) (FIG. 19) when bound to the inhibitor.

Inhibitor Binding.

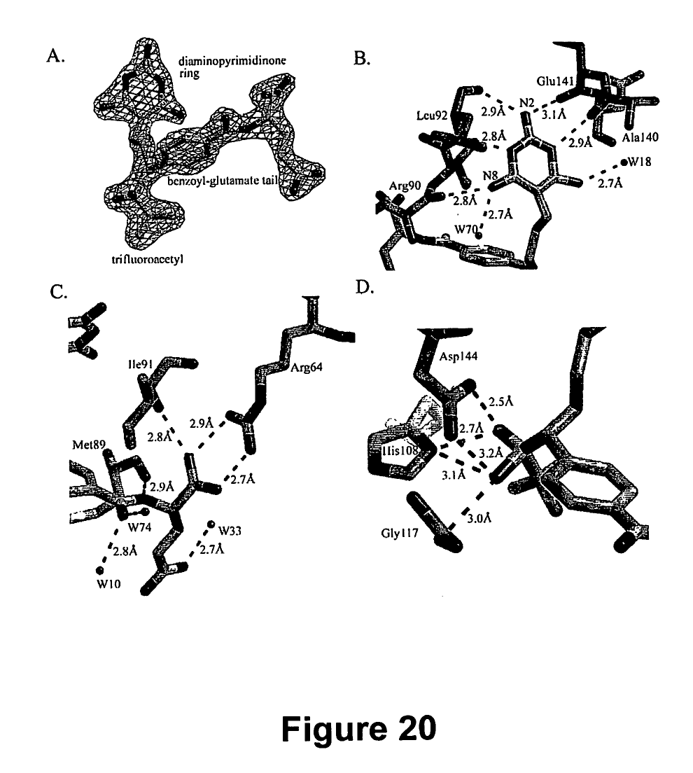

The cofactor binding pocket of GAR Tfase is located at the interface between the N-terminal mononucleotide binding domain and the C-terminal half of the structure (FIG. 18). Only the R form of compound 10-CF₃CO-DDACTHF is found in the folate-binding site (FIG. 18), as compared to the complex of 10-formyl-TDAF with E. coli GAR Tfase and substrate (PDB code 1C2T), in which both R and S diastereomers can be modeled into the electron density (Greasley, S. E., et al., Biochemistry 1999, 38, 16783-16793). The binding site for the folate cofactor moiety consists of three parts: the pteridine binding cleft, the benzoylglutamate region, and the formyl transfer region (FIG. 20).

The Pteridine Binding Cleft.

The diaminopyrimidinone ring of 101 is deeply buried in the active site cleft and occupies the same location as the quinazoline ring of 10-formyl-TDAF (103) in E. coli GAR Tfase complex (PDB code 1C2T). The connecting stem from the diaminopyrimidinone ring, composed of single carbon bonds, is longer than its counterpart in 10-formyl-TDAF (103), due to the removal of the fused benzene ring (FIG. 20), that makes it more flexible when adapting to the binding site in order to optimize the gem-diol interactions with the protein. The diaminopyrimidinone ring of 101 is tilted about 15° relative to the quinazoline ring of 103, which places N2 within the hydrogen bonding range (3.1 Å) of the backbone carbonyl oxygen of Glu141 (FIG. 20). The diaminopyrimidinone ring conserves all of the key interactions that were previously observed with the quinazoline ring of 103, and provides additional key hydrogen bonds with the enzyme. Several hydrophobic residues encircle a deep cavity holding the heterocycle. The hydrophobic pocket consists of Leu85, Ile91, Leu92, Phe96 and Val97 lining one end and the folate-binding loop 141-146 at the other. The diaminopyrimidinone ring makes six hydrogen bonds to the main-chain amides and carbonyls of Arg90, Leu92, Ala140, Glu141 and Asp144, and two hydrogen bonds to ordered waters (W18 and W70) (FIG. 20).

In the quinazoline ring of 10-formyl-TDAF (103), the N8 of the folate pteridine ring is replaced by a carbon. This nitrogen has been proposed to play a key role in recognition and interaction with folate-binding enzymes and forms one end of an H-bond donor-acceptor-donor array. While its replacement with carbon does not preclude the binding to GAR Tfase, its presence appears to contribute to substrate recognition by the folate transport system and/or FPGS. The diaminopyrimidinone ring of 101, however, preserves this nitrogen (N8) and, consequently, exhibits superb biological properties. In the 10-CF₃CO-DDACTHF complex, this amino group forms hydrogen bonds to the carbonyl oxygen of Arg90 (2.8 Å) and an ordered solvent molecule W70 (2.7 Å) (FIG. 20).

Glutamate Tail:

The role of the benzoylglutamate group of the folate is not yet fully understood. However, the 10-CF₃CO-DDACTHF 101 compound without the benzoylglutamate tail is inactive against both GAR Tfase and AICAR Tfase. In the 10-CF₃CO-DDACTHF complex, the p-aminobenzoate moiety is located in a hydrophobic pocket and sandwiched between the side chains of Ile91 and Ser 118. The electron density of the carbonyl group is well defined and in the same plane as the phenyl ring. The glutamate tail is oriented almost perpendicular to the p-aminobenzoate plane and parallel to the aliphatic stem of the diaminopyrimidinone ring (FIG. 20).

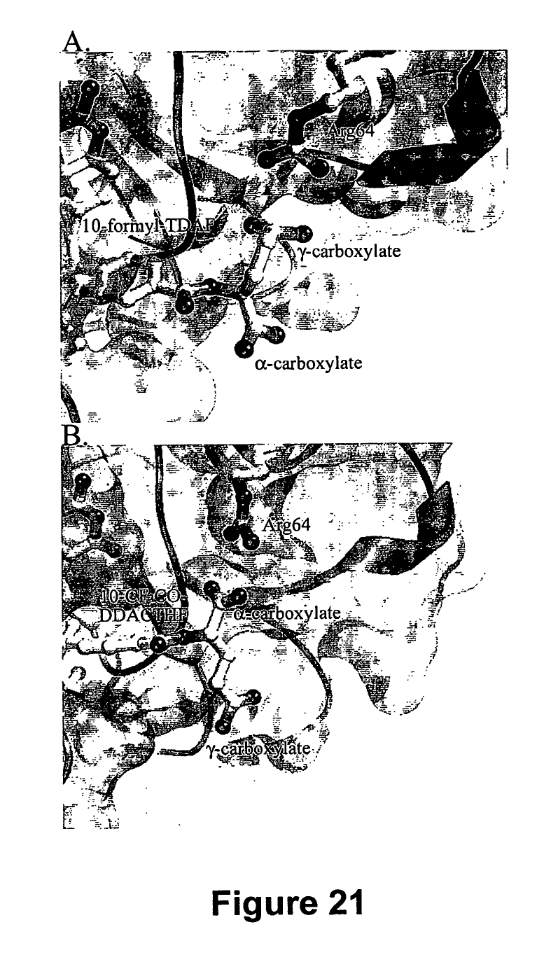

The glutamate moiety is solvent exposed, as expected, but exhibits a remarkably well ordered structure (FIG. 20), in contrast to its flexibility in E. coli GAR Tfase complex structures. A single glutamate can contribute substantially to tight binding as indicated by the lack of inhibition of analogue 101 without the glutamate (compound 111) for rhGAR Tfase (data not shown). The glutamate of 101 in this complex may then reflect its preferred location in the same surface pocket, as found in previous folate analogue complexes with E. coli GAR Tfase (FIG. 21).

In E. coli, two distinct polyglutamation activities involve amide linkage through either α or γ carboxylate (Ferone, R., et al., J. Biol. Chem. 1986, 261, 16363-16371; and Ferone, R., et al., J. Biol. Chem. 1986, 261, 16356-16362). Either of these glutamate positions could be anchored via a salt bridge to Arg64, so that the branching carboxyl points out of the binding pocket; thus additional glutamate moieties at either the a or g position can be easily accommodated. In the BW1476U89 (Klein, C., et al., J. Mol. Biol. 1995, 249, 153-175) and 10-formyl-TDAF (103) complexes (Greasley, S. E., et al., Biochemistry 1999, 38, 16783-16793), the γ-glutamate carboxylate forms a salt bridge with Arg64, and the α-carboxylate points out, reflecting the inhibitor conformation for α-polyglutamated analogues (FIG. 21). However, in the epoxide-derived folate analogue complex structure (PDB code 1JKX) (Greasley, S. E., et al., Biochemistry 2001, 40, 13538-13547), the α-glutamate carboxylate forms the salt bridge with the E. coli enzyme instead, showing the preferred interaction with the γ-polyglutamated conformer. In some cases, no obvious preferred binding orientation is observed, therefore, the glutamate tail tends to becomes disordered in these crystal structures, as in the DATHF and E. coli GAR Tfase complex (Almassy, R. J., et al., Proc. Natl. Acad. Sci. U.S.A. 1992, 89, 6114-6118).

This issue is simplified in eukaryotes where all the glutamates are added at the γ carboxylate of the previous glutamate. The glutamate tail of the 10-CF₃CO-DDACTHF in the human GAR Tfase complex displays unambiguous density (FIG. 20). A salt bridge (2.7 Å) is formed (FIG. 20) between the glutamate α-carboxylate and Arg64, so that the γ-carboxylate points to the solvent (FIG. 21). An additional interaction observed here includes a hydrogen bond between the Ile91 backbone amide and the α-glutamate carboxylate (2.8 Å) (FIG. 20).

Formyl Transfer Region and the Gem-Diol Structure:

Key interactions for tight binding of inhibitor 101 to GAR Tfase are found in the formyl transfer region. Strong density next to the ketone oxygen indicates that the ketone is hydrated to a gem-diol, similar to the 10-formyl-TDAF and β-GAR complex with the E. coli GAR Tfase (PDB code 1C2T).

The gem-diol forms extensive interactions with the formyl transfer region, especially with Asp144 and His108, two essential residues in the formyl transfer reaction (FIG. 20). The Asp144 carboxylate hydrogen bonds (2.5 Å and 2.7 Å) to each of the hydroxyl groups of the gem-diol. The N3 in the imidazole ring of His108 also forms hydrogen bonds with both hydroxyls of the gem-diol (OA1 (2.7 Å) and OA2 (3.1 Å)). Additionally, OA2 also makes a potential hydrogen bond (3.0 Å) with the backbone carbonyl oxygen of Gly117. This extensive hydrogen bonding interaction between the enzyme and the inhibitor explains why the corresponding alcohol (FIG. 13, compound 102) of the 10-CF₃CO-DDACTHF, which lacks one of the hydroxyl groups, is ca. 50 times less potent.

Folate-Binding Loop.

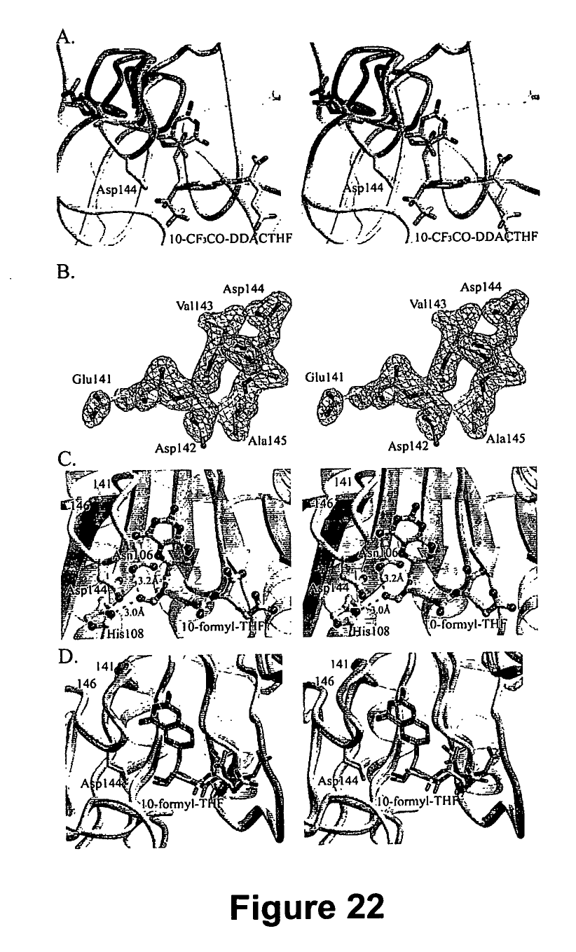

One of the main challenges of inhibitor design for GAR Tfase is the structural isomerism of the folate-binding loop 141-146, which contains a key residue (Asp144) for formyl transfer. Under different conditions, this loop shows a variety of conformations (Klein, C., et al., J. Mol. Biol. 1995, 249, 153-175; Greasley, S. E., et al., Biochemistry 1999, 38, 16783-16793; Zhang, Y., et al., Biochemistry 2002, 41, 14206-14215; Greasley, S. E., et al., Biochemistry 2001, 40, 13538-13547; and Su, Y., et al., J. Mol. Biol. 1998, 281, 485499) (FIG. 22). For example, in the obligate monomeric mutant (E70A) of E. coli GAR (PDB code 2GAR) at pH 3.5, this loop folds into the folate-binding pocket (closed conformation) and occupies the folate binding position (FIG. 22), whereas at pH 7.5 (PDB code 3GAR) (Su, Y., et al., J. Mol. Biol. 1998, 281, 485-499), it moves away so that the binding pocket is accessible to folate or folate analogues (FIG. 22). In the E coli GAR Tfase complex with 10-formyl-TDAF (103) and substrate (PDB code 1C2T) (Greasley, S. E., et al., Biochemistry 1999, 38, 16783-16793), the folate-binding loop stabilizes the gem-diol structure through hydrogen bonds between Asp144 and the hydrated aldehyde. In an epoxide-derived analogue complex (PDB 1JKX), the loop has yet another conformation (FIG. 22) where Asp144 interactions are mediated via a cluster of ordered solvent molecules (Greasley, S. E., et al., Biochemistry 2001, 40, 13538-13547), instead of direct hydrogen bonds to the inhibitor.

In unliganded human GAR Tfase (PDB code 1MEJ) at pH 8.5, this loop is “half-open”, but at pH 4.2 (PDB code 1 MEO), these residues are disordered (Zhang, Y., et al., Biochemistry 2002, 41, 14206-14215) (FIG. 22). These multiple conformations in which residues 141-146 have very high B values, or are disordered, make it problematic to determine which structure is optimal for computational calculations and docking studies. However, this flexible loop is stabilized in the human GAR Tfase upon the binding of the folate-analog, 10-CF₃CO-DDACTHF (FIG. 22). The loop exhibits excellent density with a comparable B value (33.8 Å²) to the overall enzyme (33.0 Å²) (FIG. 19), and has an identical conformation to the E. coli enzyme complex with 10-formyl TDAF, except for the orientation of the side chains of Glu141, Asp142 and Val143.

These changes in the loop conformation help provide most of the inhibitor interactions with enzyme. The backbone carbonyl oxygens of Glu141 and Asp144 are involved in directly anchoring the diaminopyrimidinone ring, whereas a water molecule (W18 in molecule A and W27 in molecule B) mediates interactions between the backbone amide and carbonyls of the folate-binding loop and the diaminopyrimidinone ring. But most importantly, Asp144 at the tip of the loop provides a key interaction with the inhibitor. The side chain of Asp144 rotates about 90° (RMSD of 5.5 Å, in comparison to the unliganded human GAR Tfase structure) and flips into the folate-binding pocket to form hydrogen bonds with the gem-diol (FIG. 22), putting it in the vicinity of His108 (FIG. 22). Contrary to the flexible Asp144, His108 is tightly anchored by its interaction with the main-chain carbonyl oxygen of Lys115 (2.8 Å) and the hydroxyl of Ser 10 (3.0 Å). The translocation of Asp144 facilitates formation of a salt bridge with His108, which appears to be essential for the formyl transfer reaction (Shim, J. H., et al., Biochemistry 1999, 38, 10024-10031). The highly ordered folate-binding loop and its extensive interactions with inhibitor suggest that this structure is an excellent template for computational docking.

Docking of Folate Cofactor.

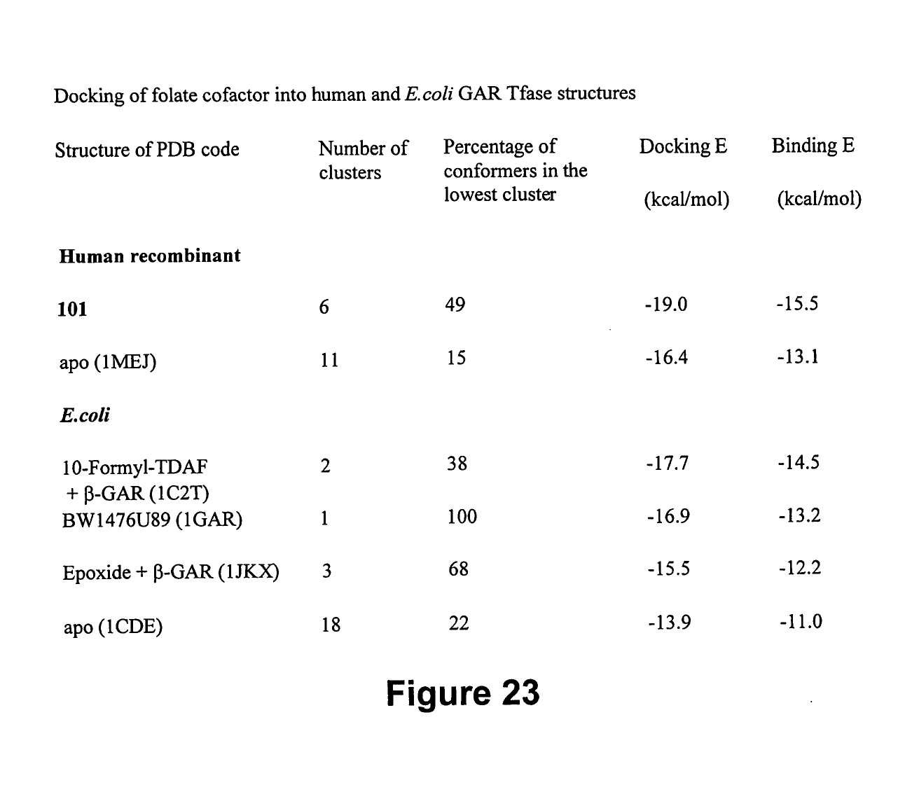

The natural cofactor 10-formyl-THF is unstable and not ideal for co-crystallization studies. Therefore, computational docking was used to probe the interaction between enzyme and natural cofactor. Using the coordinates of the human GAR Tfase complex with 10-CF₃CO-DDACTHF, six docking clusters were obtained. The lowest energy cluster with a docking energy −19.0 kcal/mol and binding energy of −15.5 kcal/mol is also the largest, representing almost half (49%) of all the conformers (FIG. 23). In contrast, as many as eleven clusters were obtained with apo human GAR Tfase (PDB code 1 MEJ), with the most dominant cluster representing only 15% of the conformers, with a much less favorable docking energy (−16.4 kcal/mol) (FIG. 23). Furthermore, when docked with the apo human GAR Tfase template, the pteridine ring is inserted between the folate binding loop (141-146) and His108, contradictory to published kinetic and structural data. In the best conformer of cofactor with rhGAR Tfase/10-CF₃CO-DDACTHF (101) complex as template, the formyl group hydrogen bonds to His108 (3.0 Å) and Asn106 (3.2 Å), with Asp144 in the vicinity, potentially stabilizing the protonation of His108 (FIG. 22). This conformation of the docked folate cofactor strongly supports the proposed mechanism (Shim, J. H., et al., Biochemistry 1999, 38, 10024-10031; and Su, Y., et al., J. Mol. Biol. 1998, 281, 485-499). The rhGAR Tfase/10-CF₃CO-DDACTHF (101) template then is much more appropriate for mechanistic studies and structural-based drug design.

In order to compare folate binding to both human and E. coli GAR Tfases, a comprehensive docking analysis was performed on four different E. coli GAR Tfase structures (FIG. 23). The lowest docking (−17.7 kcal/mol) and binding energy cluster (−14.5 kcal/mol) was observed with the E. coli GAR Tfase complex with 10-formyl-TDAF (103) and substrate β-GAR (PDB code 1C2T). With the multisubstrate adduct complex (PDB code 1GAR), BW1476U89, only one cluster was obtained. However, the covalent carbon and sulfur linker between the folate and substrate moieties of the inhibitor may distort the active site resulting in less favorable docking energy (DockingE=−16.9 kcal/mol). Similarly, the epoxide-derived inhibitor complex (PDB code 1 JKX) is also less favorable as a template (DockingE=−15.5 kcal/mol). The worst case was found for apo E. coli GAR Tfase, with a scattering of 18 clusters, none of which has a reasonable folate-binding position, with the docking energy of only −13.9 kcal/mol for the lowest energy cluster. In this case, the docked folate pteridine ring reverses its position and binds to the substrate-binding pocket, which obviously contradicts the folate analogue and E. coli GAR Tfase complex structures.

Comparison of the best docking results from the various human and E. coli GAR Tfase structures (template from rhGAR Tfase/10-CF₃CO-DDACTHF and eGAR Tfase/10-formyl-TDAF-β-GAR, respectively) shows that the folate-binding site and the folate cofactor have similar conformations (FIG. 22). Slight differences (RMSD of 0.4 Å at 140-146) are mostly caused by the higher flexibility of this loop in the E. coli structures. For the folate cofactor, the pteridine ring forms hydrogen bonds mainly to the protein backbone. The formyl group is close to His108, Asp144 and Asn106, the formyl transfer catalytic triad (FIG. 22). The docking confirms that His108, Asp144 and Asn106 are central to the enzyme formyl transfer reaction.

Materials and Methods

Materials.

Luria broth and agar were obtained from Life Technologies (Gaithersburg, Md.). All common buffers and reagents were purchased from Sigma-Aldrich Corp. (St. Louis, Mo.).

### Synthesis and Characterization of 10-CF₃CO-DDACTHF (1)

### Methyl 4-(3,3,3-trifluoro-2-oxopropyl)benzoate (106)

Known acid chloride 105 (37.7 g, 177 mmol) was dissolved in anhydrous CH₂Cl₂ (500 mL) and cooled to −60° C. Trifluoroacetic anhydride (77.0 mL, 543 mmol, 3 equiv) was added slowly to the stirring solution. Anhydrous pyridine (30.0 mL, 371 mmol, 2 equiv) was added dropwise and the reaction mixture was stirred at 60° C. for 4 h. The reaction was quenched by the dropwise addition of H₂O (35 mL) to the stirring solution, followed by warming to 25° C. The reaction mixture was partitioned between H₂O (600 mL) and CH₂Cl₂ (100 mL). The organic layer was washed with 1 N HCl (2×500 mL) and saturated aqueous NaCl (500 mL) followed by concentration under reduced pressure. Chromatography (SiO₂, 1:1 hexanes/EtOAc) afforded 106 (41.3 g, 95%) as a yellow oil: MALDIFTMS (DHB) m/z 245.0436 (M−H⁺, C₁₁H₉F₃O₃ requires 245.0431).

### Methyl 4-(3,3,3-trifluoro-2-dimethylhydrazonopropyl)benzoate (107)

Compound 106 (35.6 g, 145 mmol) was dissolved in anhydrous EtOH (600 mL). N,N-Dimethylhydrazine (55.0 mL, 724 mmol, 5 equiv) was added to this solution followed by glacial acetic acid (8.30 mL, 145 mmol, 1 equiv) and the mixture was stirred at 25° C. for 48 h. The reaction mixture was concentrated under reduced pressure. Chromatography (SiO₂, 2:1 hexanes/EtOAc) afforded 107 (26.8 g, 64%) as a yellow oil: MALDIFTMS (DHB) m/z 289.1166 (M+H⁺, C₁₃H₁₅F₃N₂O₂ requires 289.1158).

### Methyl 4-[4-bromo-1-(2,2,2-trifluoro-1-dimethylhydrazonoethyl)butyl]benzoate (108)

NaH (60% dispersion, 2.34 g, 58.6 mmol, 1 equiv) was added to a stirred solution of 107 (16.7 g, 58.1 mmol) in anhydrous DMF (250 mL) at 0° C. The solution was stirred at 0° C. for 15 min. 1,3-dibromopropane (35.0 mL, 345 mmol, 6 equiv) was added quickly to the reaction and the cooling bath was removed. The reaction mixture was stirred at 25° C. for 2.5 h. The reaction was quenched by the addition of saturated aqueous NH₄Cl (150 mL). The reaction mixture was partitioned between EtOAc (600 mL) and H₂O (400 mL). The organic layer was washed with H₂O (2×500 mL) and saturated aqueous NaCl (1×500 mL) followed by concentration under reduced pressure. Chromatography (SiO₂, 4:1 hexanes/EtOAc) afforded 108 (15.4 g, 65%) as a yellow oil: MALDIFTMS (DHB) m/z 409.0723 (M+H⁺, C₁₆H₂₀BrF₃N₂O₂ requires 409.0733).

### Methyl 4-[4-cyano-4-ethoxycarbonyl-1-(2,2,2-trifluoro-1-dimethylhydrazono-ethyl)-butyl]benzoate (109)

A suspension of NaH (60% dispersion, 26.0 g, 655 mmol, 18 equiv) in anhydrous DMF (300 mL) at 0° C. was treated dropwise with ethyl cyanoacetate (70.0 mL, 657 mmol, 18 equiv). The reaction mixture was stirred at 0° C. for 30 min, forming the sodium salt as a clear solution. This anion was treated with a solution of 108 (14.4 g, 35.7 mmol) in anhydrous DMF (300 mL). The reaction mixture was stirred at 25° C. for 2 h before being quenched by the addition of saturated aqueous NH₄Cl (50 mL). The reaction mixture was diluted with EtOAc (600 mL) and washed with H₂O (5×400 mL) and saturated aqueous NaCl (400 mL). The organic layer was dried (Na₂SO₄), filtered, and concentrated under reduced pressure. The excess ethyl cyanoacetate was distilled off and the residual product was purified by chromatography (SiO₂, 7:1 hexanes/EtOAc) affording 109 (11.2 g, 71%) as a yellow oil: MALDIFTMS (DHB) m/z 464.1768 (M+Na⁺, C₂₁H₂₆F₃N₃O₄Na requires 409.1768).

### Methyl-4-[4-(2,4-diamino-6-oxo-1,6-dihydropyrimidin-5-yl)-1-(2,2,2-trifluoro-1-di-methylhydrazonoethyl)butyl]benzoate (110)

Sodium metal (0.71 g, 30.9 mmol, 2 equiv) was added to anhydrous CH₃OH (15 mL) and the reaction mixture was stirred at 25° C. for 10 min to generate NaOCH₃. Guanidine-HCl (1.47 g, 15.4 mmol, 1 equiv) was added and the reaction mixture was stirred at 25° C. for 30 min. Separately, 109 (6.78 g, 15.4 mmol) was dissolved in anhydrous CH₃OH (15 mL) and this solution was added quickly to the stirring reaction mixture. The resulting reaction mixture was stirred at reflux for 16 h. The reaction mixture was applied directly to a SiO₂ plug. Impurities were removed by washing with 3:1 hexanes/EtOAc. The product was subsequently eluted by washing with 10:1 CHCl₃/CH₃OH to afford 110 (3.91 g, 56%) as a tan solid: MALDIFTMS (DHB) m/z 455.2001 (M+H⁺, C₂₀H₂₅F₃N₆O₃ requires 455.2013).

### 4-[4-(2,4-diamino-6-oxo-1,6-dihydropyrimidin-5-yl)-1-(2,2,2-trifluoro-acetyl)butyl]benzoic acid (111)

A solution of 110 (2.11 g, 4.64 mmol) in 3:1 CH₃OH—H₂O (80 mL) was treated with LiOH-1H₂O (0.59 g, 13.9 mmol, 3 equiv) and the reaction was stirred at 25° C. for 24 h. The reaction mixture was diluted with H₂O (100 mL) and the aqueous layer was washed with EtOAc (100 mL). The aqueous layer was acidified to pH=4 by the addition of 1 N aqueous HCl. The reaction mixture was concentrated under reduced pressure and the residue was treated with MeCN (3×100 mL) to remove traces of H₂O to provide 111 (1.84 g, 100%) which was used without further purification: MALDIFTMS (DHB) m/z 399.1275 (M+H⁺, C₁₇H₁₇F₃N₄O₄ requires 399.1275).

### Di-tert-butyl N-{4-[4-(2,4-diamino-6-oxo-1,6-dihydropyrimidin-5-yl)-1-(2,2,2-trifluoro-acetyl)butyl]benzoyl}-L-glutamate (112)

A solution of 111 (1.84 g, 4.62 mmol) and di-tert-butyl L-glutamate hydrochloride (1.71 g, 4.76 mmol, 1 equiv) in DMF (20 mL) was treated with NaHCO₃ (1.41 g, 16.8 mmol, 4 equiv) followed by EDCI (1.71 g, 8.9 mmol, 2 equiv). The reaction mixture was stirred at 25° C. for 48 h before the solvent was removed under reduced pressure. The resulting residue was suspended in CHCl₃ (300 mL) and washed with saturated aqueous NaHCO₃ (2×300 mL). The organic layer was dried (Na₂SO₄), filtered, and concentrated under reduced pressure. Chromatography (SiO₂, 10:1 CHCl₃/CH₃OH) afforded 112 (1.29 g, 44%) as a yellow solid: MALDIFTMS (DHB) m/z 640.2975 (M+H⁺, C₃₀H₄₀F₃N₅O₇ requires 640.2952).

### N-{4-[4-(2,4-Diamino-6-oxo-1,6-dihydropyrimidin-5-yl)-1-(2,2,2-trifluoroacetyl)butyl]benzoyl}-L-glutamic acid (101)

A solution of 112 (1.29 g, 2.02 mmol) in CHCl₃ (120 mL) cooled to 0° C. was treated with trifluoroacetic acid (35 mL). The reaction mixture was allowed to warm and stirred at 25° C. for 12 h. The reaction was concentrated under reduced pressure. Et₂O (75 mL) was added and a precipitate formed. The precipitate was collected, triturated with Et₂O (3×75 mL) and dried in vacuo to give 101-CF₃CO₂H (1.30 g, 100%) as a white solid: MALDIFTMS (DHB) m/z 528.1709 (M+H⁺, C₂₂H₂₄F₃N₅O₇ requires 528.1701).

### N-{4-[4-(2,4-Diamino-6-oxo-1,6-dihydropyrimidin-5-yl)-1-(2,2,2-trifluoro-1-hydroxy-ethyl)butyl]benzoyl}-L-glutamic acid (102)

A solution of 112 (0.010 g, 0.016 mmol) in anhydrous CH₃OH (0.5 mL) at −20° C. was treated with NaBH₄ (0.0012 g, 0.031 mmol, 2.0 equiv). The reaction mixture was stirred at −20° C. for 30 min before it quenched by the addition of H₂O (1 mL). The mixture was diluted with EtOAc (5 mL) and washed with H₂O (2×1 mL). The organic layer was dried (Na₂SO₄), filtered, and concentrated under reduced pressure. The resulting product (0.009 g, 0.014 mmol) was treated with 4 N HCl-dioxane (2 mL) at 0° C. and the solution was allowed to warm and stir at 25° C. for 3 h. The reaction was purged with N₂ and then concentrated under reduced pressure. Et₂O (1 mL) was added and a precipitate formed. The precipitate was collected, triturated with Et₂O (3×1 mL), and dried in vacuo to give 102-HCl (0.004 g, 90% from 101) as a yellow solid: MALDIFTMS (DHB) m/z 530.1838 (M+H⁺, C₂₂H₂₆F₃N₅O₇ requires 530.1857).

Recombinant Human GAR Tfase Protein Preparation.

The recombinant human GAR Tfase (purN) construct includes residues 808-1010 from the human trifunctional enzyme (purD-purM-purN). The gene was subcloned into the pet22b vector using the Ndel/Xhol cloning site with ahexa-histidine tag at the C-terminus. The plasmid was transformed into the E. coli expression strain BL21 (DE3) Gold. The protein was expressed and purified as described previously (Zhang, Y., et al., Biochemistry 2002, 41, 14206-14215). The yield of the protein is greater than 30 mg per liter LB broth after purification, with at least 98% purity when assessed by SDS-PAGE. The purified protein was used in the inhibition assays, cytotoxic assays and crystallization experiments.

GAR Tfase Inhibition Assay.

The Kᵢ values for the folate analogues were measured as previously described (Boger, D. L., et al., Bioorg. Med. Chem. 1997, 5, 1817-1830). Briefly, each compound was dissolved in dimethyl sulfoxide (DMSO) and then diluted in assay buffer. The low concentration of DMSO did not affect enzyme activity. Thus, all assays were conducted by mixing 10 μM 10-formyl-5,8-dideazafolate (fDDF), 20 μM inhibitor in total volume of 1 mL buffer (0.1 M HEPES, pH 7.5) at 26° C., and the reaction initiated by the addition of 76 nM E. coli or human GAR Tfase. The assay monitors the deformylation of fDDF (Δε=18.9 mM⁻¹ cm⁻¹ at 295 nm) resulting from the transfer of the formyl group to β-GAR. If the inhibitor was found to be active, a series of 1/vᵢ versus 1/[GAR] at different, fixed concentrations of I (e.g. 4,8,12,16,20,32 μM) were generated in order to determine the Kᵢ using the Michaelis-Menton equation for competitive inhibition.

Cytotoxic Assay.

The cytotoxic activity of the compounds was measured using CCRF-CEM human leukemia cells, as described previously (Boger, D. L., et al., Bioorg. Med. Chem. 1997, 5, 1847-1852). Two mutant cell lines, CCRF-CEM/MTX and CCRF-CEM/FPGS⁻ were used to determine the dependence on the reduced folate active transport system and folylpolyglutamate synthetase (FPGS), respectively (Jansen, G., et al., Cancer Res. 1989, 49, 2455-2459).

Crystallization and Data Collection.

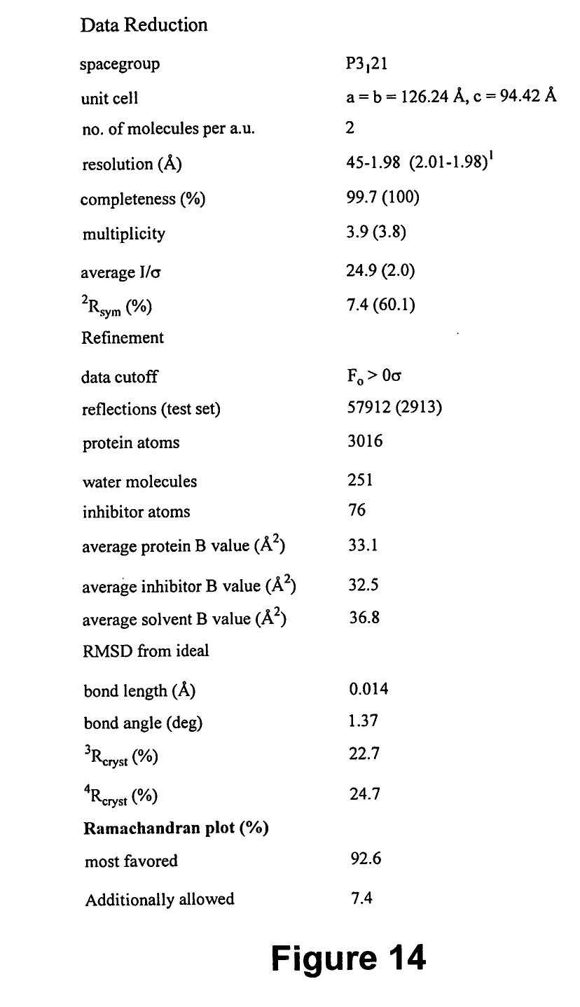

Crystals of human GAR Tfase in complex with 10-CF₃CO-DDACTHF (101) were obtained by the method of vapor diffusion in 2 μL sitting drops. The protein solution, at a concentration of 16 mg/mL, was mixed with 3-fold molar excess of the inhibitor. Needle-shaped crystals were obtained after 7 days at 4° C. from a solution of PEG4K, 0.2 M ammonium sulfate, 50 mM HEPES, pH 6.7-7.0. Data were collected on an ADSC 2×2 CCD detector from a single crystal, cryoprotected by 20% glycerol at −179° C. on beam-line 9-2 at the Stanford Synchrotron Radiation Laboratory (SSRL). The data set was processed with HKL2000 (Otwinowski, Z., et al., Methods Enzymol. 1997, 276, 307-326). The crystal spacegroup is trigonal P3₁21 with two molecules per asymmetric unit with a Matthews coefficient (Matthews, B. W., J. Mol. Biol. 1968, 33, 491-497) of 4.5 Å³Da⁻¹, corresponding to a relatively high solvent content of 75%, consistent with the rather fragile crystals. The statistics for the data collection and processing are summarized in FIG. 14.

The human GAR Tfase complexed with 10-CF₃CO-DDACTHF (101) was also crystallized in MPEG5500, 100 mM sodium acetate at pH 5.0-5.5 in the trigonal spacegroup P3, with a similar unit cell (a=b=126.07 Å, c=94.02 Å) to the pH 7.0 crystals (FIG. 14), but four molecules per asymmetric unit (Vₘ=4.5 Å³ Da⁻¹).

Structure Solution and Refinement

The crystal structure of human GAR Tfase in complex with 10-CF₃CO-DDACTHF (101) was determined by molecular replacement (MR) (Rossmann, M. G., The Molecular Replacement Method, 1972, Gordon & Breach, New York) using unliganded human GAR Tfase (PDB code 1MEJ) as the search model in the program AmoRe from the CCP4 package (CCP4, Acta Crystallogr. 1994, D50, 760-763). The initial refinement was carried out using the program CNS (Marangos, P. J., et al., Epilepsia 1990, 31, 239-246). The location of the folate inhibitor was clear in Fo-Fc maps even after the first round of refinement. The inhibitor model was built into the electron density using 0 (Jones, T. A., et al., Acta Crystallogr. 1991, A47, 110-119); strong density adjacent to the ketone oxygen suggested the hydrated form of the inhibitor was bound. Two-fold non-crystallographic restraints were used in the refinement of molecules A and B, except for flexible regions (residues 21-26, 58-63, 141-146 and 190-200) that have been seen to differ in other GAR Tfase structures The final refinement was carried out using TLS refinement (Winn, M. D., et al., Acta Crystallogr. 2001, D57, 122-133) from the CCP4 Refmac program. The final R_{cryst} and R_{free} are 22.3% and 24.7% respectively. The final model was evaluated by Procheck (Laskowski, R. A., et al., J. Appl. Crystallogr. 1993, 26, 283-291) and has 92.6% of the residues in the most favored regions of the Ramachandran plot with no outliers. FIGS. 18, 20 and 22 were created with Bobscript (Esnouf, R. M., J. Mol. Graph. Model 1997, 15, 132-134) and rendered with Raster3D (Merritt, E. A., et al., Acta Crystallogr. 1994, D50, 869-873). FIG. 21 was generated with PMV (Coon, S. I., et al., in Ninth Annual International Python Conference, 2001, Long Beach, Calif., U.S.A.). The final refinement statistics are presented in FIG. 14. Coordinates and structure factors have been deposited in the PDB (Berman, H. M., et al., Nucleic Acids Research 2000, 28, 235-242) with accession code 1NJS.

Automated Docking of Cofactor.

Two human and four E. coli templates of GAR Tfase for computational docking of the cofactor 10-formyl-THF were extracted from apo and ligand complex structures excluding the inhibitor coordinates. For human recombinant GAR Tfase, the published apo structure at pH 8.5 (PDB code 1MEJ) and its complex with 101 were used for computer docking studies. For E coli GAR Tfase, the apo structure at pH 7.5 (PDB code 1CDE), and its complexes with BW1476U89 (PDB code 1GAR), an epoxide-based inhibitor and substrate (PDB code 1JKX), and 10-formyl-TDAF (103) and substrate (PDB code 1C2T), were used for cofactor docking. Non-polar hydrogens were merged with heavy atoms and Kollman charges were assigned (Weiner, S. J., et al., J. Am. Chem. Soc. 1984, 106, 765-784). His108 was fully protonated with charge +1 due to its reported high PKₐ (Shim, J. H., et al., Biochemistry 1998, 37, 8776-8782). 10-formyl-THF was built and minimized with INSIGHTII [Molecular Simulations, Inc.]. All-atom Gasteiger charges were added and non-polar hydrogens merged (Gasteiger, J., et al., Tetrahedron 1980, 36, 3219-3228). The native cofactor 10-formyl-THF was docked into the active site using AutoDock 3.0.5 (Goodsell, D. S., et al., Proteins 1990, 8, 195-202; and Morris, G. M., et al., J. Computatinal Chemistry 1998, 19, 1639-1662), a suite of programs for automated docking of flexible ligands into protein targets. The docking simulations were performed using the Lamarkian genetic algorithm with a rapid grid-based lookup method for energy evaluation. The Auto Tors utility of AutoDock was used to assign eleven rotatable bonds and ten aromatic carbons in the ligand. Parameters for the docking were as follows: trials of 100, population size of 150, random starting position and conformation, translation step of 0.5 Å, rotation step of 15°, elitism of 1, mutation rate of 0.02, crossover rate of 0.8, local search rate of 0.06, and 50 million energy evaluations. Final docked conformations were clustered using a tolerance of 1.5 Å root-mean-square deviation (RMSD).

Abbreviations:

GAR Tfase, glycinamide ribonucleotide transformylase; 10-CF₃CO-DDACTHF, 10-trifluoroacetyl-5,10-dideaza-acyclic-5,6,7,8-tetrahydrofolic acid; AICAR Tfase, 5-aminoimidazole-4-carboxamide-ribonucleotide transformylase; 10-formyl-THF, 10-formyl-tetrahydrofolic acid; β-GAR, β-glycinamide ribonucleotide; DHFR, dihydrofolate reductase; DDATHF, 5,10-dideaza-5,6,7,8-tetrahydrofolic acid; FPGS, folylpolyglutamate synthetase; 10-formyl-TDAF, 10-formyl-5,8,10-trideazafolic acid; 10-formyl-DDACTHF, 10-formyl-5,10-dideaza-acyclic-5,6,7,8-tetrahydrofolic acid; TS, thymidylate synthetase; fDDF, 10-formyl-5,8-dideazafolic acid; DMSO, dimethyl sulfoxide; SSRL, Stanford Synchrotron Radiation Laboratory; RMSD, root-mean-square deviation; MR, molecular replacement.

What is claimed is:

1. A compound represented by the following structure: 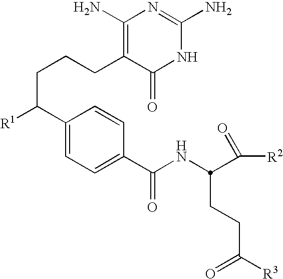
wherein:
R¹ is a radical selected from the group consisting of —C(O)H, —CH₂OH, —CH═NNMe₂, —C(O)CF₃, and —CH(OH)CF₃;
R² is a radical selected from the group consisting of —OH, —OtBu, glutamyl, and oligoglutamyl;
R³ is a radical selected from the group consisting of —OH, —OtBu, glutamyl, and oligoglutamyl;
each glutamyl being independently represented by the formula: —NHCH(C(O)R⁴)(CH₂)₂C(O)R⁵ wherein R⁴ and R⁵ are each radicals independently selected from the group consisting of —OH and —OtBu;
each oligoglutamyl having at least one terminal glutamyl and between one and four non-terminal glutamyl residues;
each terminal glutamyl being independently represented by the formula —NHCH(C(O)R⁴)(CH₂)₂C(O)R⁵ wherein R⁴ and R⁵ are each radicals independently selected from the group consisting of —OH and —OtBu;
each non-terminal glutamyl being independently represented by the formula —NHCH(C(O)R⁶)(CH₂)₂C(O)R⁷ wherein R⁶ and R⁷ are each radicals independently selected from the group consisting of —OH, —OtBu, terminal glutamyl, and non-terminal glutamyl;
with a proviso that at least one of R⁶ and R⁷ is either terminal glutamyl or non-terminal glutamyl.

2. A compound according to claim 1 represented by the following structure:

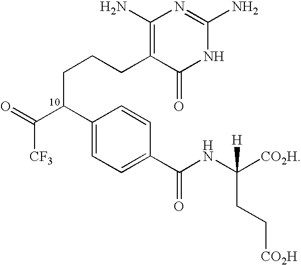

3. A compound according to claim 1 represented by the following structure:

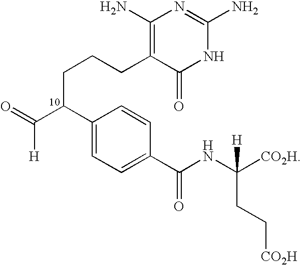

4. A compound according to claim 1 represented by the following structure: 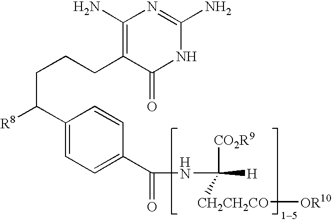
wherein
R⁸ is a radical selected from the group consisting of —C(O)H and —C(O)CF₃; and
R⁹ and R¹⁰ are each a radical independently selected from the group consisting of —H and -tBu.

5. A compound according to claim 1 represented by the following structure: 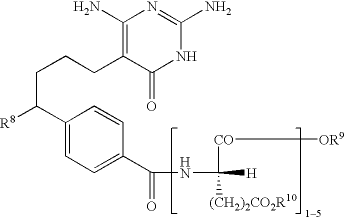
wherein
R⁸ is a radical selected from the group consisting of —C(O)H and —C(O)CF₃; and
R⁹ and R¹⁰ are each a radical independently selected from the group consisting of —H and -tBu.

6. A process for inhibiting glycinamide ribonucleotide transformylase comprising the step of contacting the glycinamide ribonucleotide transformylase with an inhibiting concentration of a compound described in claims 1-5.

7. A process for inhibiting aminoimidazole carboxamide ribonucleotide transformylase comprising the step of contacting the aminoimidazole carboxamide ribonucleotide transformylase with an inhibiting concentration of a compound described in claims 1-5.

8. A complex comprising glycinamide ribonucleotide transformylase and a compound of claims 1-5 wherein the compound of claims 1-5 is non-covalently complexed with the glycinamide ribonucleotide transformylase.
